(19) United States
(12) Patent Application Publication
(10) Pub. No.: US 2013/0296288 A1
(43) Pub. Date: Nov. 7, 2013

(54) ORGANIC ELECTROLUMINESCENT DEVICE

(71) Applicants: Junyou Pan, Frankfurt am Main (DE); Philipp Stoessel, Frankfurt am Main (DE)

(72) Inventors: Junyou Pan, Frankfurt am Main (DE); Philipp Stoessel, Frankfurt am Main (DE)

(73) Assignee: Merck Patent GmbH, Darmstadt (DE)

(21) Appl. No.: 13/994,766

(22) PCT Filed: Nov. 24, 2011
(86) PCT No.: PCT/EP2011/005917

§ 371 Date: Jun. 17, 2013

(30) Foreign Application Priority Data
Dec. 23, 2010 (DE) 10 2010 055 902.4

(51) Int. Cl. A61K 41/00; C09K 11/06

(52) U.S. Cl. CPC A61K 41/0057; C09K 11/06
USPC 514/186; 546/4; 548/219; 546/271.4; 252/301.16; 252/586; 514/375; 514/320

(57) ABSTRACT

The present invention relates to a salt in which at least one cation and at least one anion is in each case an emitter compound or a dye compound, characterised in that at least one of the emitter compounds is a fluorescent emitter compound. In addition, the present invention also relates to a process for the preparation of the salt according to the invention, to the use of the salt in an electronic device, and to a formulation and an electronic device which comprises the salt.

The present invention relates to a salt in which both at least one cation and also at least one anion is an emitter compound or a dye compound, characterised in that one emitter compound is a fluorescent emitter compound. In addition, the present invention also relates to a process for the preparation of the salt according to the invention, to the use of the salt in an electronic device, and to a formulation and an electronic device which comprises the salt.

Electronic devices which comprise organic, organometallic and/or polymeric semiconductors are being used ever more frequently in commercial products or are just about to be introduced onto the market. Examples which may be mentioned here are organic-based charge-transport materials (in general triarylamine-based hole transporters) in photocopiers and organic or polymeric light-emitting diodes (OLEDs or PLEDs) in display devices or organic photoreceptors in copiers. Organic solar cells (O-SCs), organic field-effect transistors (O-FETs), organic thin-film transistors (O-TFTs), organic integrated circuits (O-ICs), organic optical amplifiers or organic laser diodes (O-lasers) are also at an advanced stage of development and may achieve major importance in the future.

Many of these electronic and opto-electronic devices have, irrespective of the particular application, the following general layer structure, which can be adapted to the particular application:
(1) substrate,
(2) electrode, frequently metallic or inorganic, but also made from organic or polymeric conductive materials;
(3) charge-injection layer or interlayer for compensation of unevenness of the electrode (“planarisation layer”), frequently made from a conductive, doped polymer,
(4) organic semiconductors,
(5) possibly a further charge-transport or charge-injection or charge-blocking layer,
(6) counterelectrode, materials as mentioned under (2),
(7) encapsulation.

The above arrangement represents the general structure of an opto-electronic device, where various layers can be combined, so that, in the simplest case, an arrangement comprising two electrodes, between which an organic layer is located, results. The organic layer in this case fulfils all functions, including the emission of light. A system of this type is described, for example, in WO 9013148 A1 based on poly(p-phenylenes).

A problem which arises in a “three-layer system” of this type is, however, the lack of a possibility to optimise the individual constituents in different layers with respect to their properties, as is solved easily, for example, in the case of SMOLEDs (“small-molecule OLEDs”) through a multilayered structure. A “small molecule OLED” consists, for example, of one or more organic hole-injection layers, hole-transport layers, emission layers, electron-transport layers and electron-injection layers as well as an anode and a cathode, where the entire system is usually located on a glass substrate. An advantage of a multilayered structure of this type consists in that various functions of charge injection, charge transport and emission can be divided into the various layers and the properties of the respective layers can thus be modified separately.

The layers in SMOLED devices are usually applied by vapour deposition in a vacuum chamber. However, this process is complex and thus expensive and is unsuitable, in particular, for large molecules, such as, for example, polymers, but also for many small molecules, which frequently decompose under the vapour-deposition conditions.

The application of layers from solution is therefore advantageous, where both small molecules and also oligomers or polymers can be processed from solution.

In the conventional process for OLED production, both by deposition from the gas phase or solution-processed, it is difficult to control the distribution of the individual components. The components are usually distributed randomly. This is undesired for some physical properties of such systems, for example in the case of so-called “double doping” in triplet systems (see Kawamura, Y.; Yanagida, S.; Forrest, S. R., “Energy transfer in polymer electro phosphorescent light emitting device with single and multiple doped luminescent layers”, J. Appl. Phys., 92 (1), 87-93, 2002). It is reported therein that a very efficient polymer (PHOLED) is produced by using poly-(9-vinylcarabazole) (PVK) as host molecule, which is doped with one or more phosphorescent cyclometallated Ir(III) complexes. It is usually assumed that energy transfer, for example by the Förster mechanism, takes place in the case of double doping.

The Förster energy transfer rate can be represented theoretically, for example, by the following formula:

Γ_{DA}1/R⁶,

where R represents the separation between donor and acceptor. This separation is usually also known as the Förster radius. In order to facilitate efficient energy transfer, for example in accordance with Förster or Dexter, it is thus necessary to position the donor and acceptor, i.e. the two emitter compounds or metal complexes, as close as possible, advantageously within the so-called Förster radius.

The fact that the two emitters are usually distributed randomly means that the requisite small separation of the two emitter molecules from one another (donor and acceptor) is not guaranteed to the full extent.

The object of the present invention therefore consisted in the provision of a system in which at least two emitter compounds have, due to electrostatic interaction, the requisite small separation from one another that is necessary for efficient energy transfer between the emitter molecules.

For this purpose, the present invention provides a salt in which both at least one cation and also at least one anion is an emitter compound or a dye compound, where one emitter compound is a fluorescent emitter compound.

In other words, the salt contains at least one cation which is an emitter compound or a dye compound and an anion which is an emitter compound or a dye compound.

In the present invention, a salt is taken to mean an ionic compound comprising cations and anions whose positive and negative charges compensate or neutralise one another. The salt according to the invention can be a simple salt comprising one type of cations and one type of anions, but it can also be a double salt, either having two or more different cations and one type of anions, or having two or more different anions and one type of cations. Besides simple salts and double salts, the salt according to the invention can also be in the form of a mixed salt through at least two different simple salts or double salts, or one simple salt and one double salt, forming a mixed crystal with one another.

Preferably, at least one of the cations of the salt according to the invention is a positively charged emitter compound or dye compound and at least one of the anions is a negatively charged emitter compound or dye compound. It is particularly preferred for all ions of the salt according to the invention to be selected from emitter compounds or dye compounds.

A fluorescent emitter compound in the sense of this invention is, regarded in general terms, a unit which emits light, preferably in the visible region, from an excited singlet state, and has a positive or negative charge. The fluorescent emitter compound can function as cation or as anion in the salt according to the invention. Particular preference is given here to singly or doubly positively or negatively charged cations or anions, particularly preferably singly positively or negatively charged cations or anions.

The fluorescent emitter compound preferably contains one of the following units: mono- or polycyclic aromatic or heteroaromatic ring systems having 5 to 60 aromatic ring atoms or also tolan, stilbene or bisstyrylarylene derivatives, each of which may be substituted by one or more radicals R. Particular preference is given here to the incorporation of 1,4-phenyl, 1,4-naphthyl, 1,4- or 9,10-anthryl, 1,6-, 2,7- or 4,9-pyrenyl, 3,9- or 3,10-perylenyl, 4,4′-biphenylyl, 4,4″-terphenylyl, 4,4′-bi-1,1′-naphthylyl, 4,4′-tolanyl, 4,4′-stilbenzyl, 4,4″-bisstyrylaryl, benzothiadiazolyl, quinoxalinyl, phenothiazinyl, phenoxazinyl, dihydrophenazinyl, bis(thiophenyl)aryl, oligo(thiophenyl), phenazinyl, rubrenyl, pentacenyl, squarinyl and quinacridonyl, which are preferably substituted, or preferably conjugated push-pull systems (systems which are substituted by donor and acceptor substituents, or systems such as squarines or quinacridones, which are preferably substituted.

A monostyrylamine is taken to mean a compound which contains one substituted or unsubstituted styryl group and at least one, preferably aromatic, amine. A distyrylamine is taken to mean a compound which contains two substituted or unsubstituted styryl groups and at least one, preferably aromatic, amine. A tristyrylamine is taken to mean a compound which contains three substituted or unsubstituted styryl groups and at least one, preferably aromatic, amine. A tetrastyrylamine is taken to mean a compound which contains four substituted or unsubstituted styryl groups and at least one, preferably aromatic, amine. The styryl groups are particularly preferably stilbenes, which may also be further substituted. Corresponding phosphines and ethers are defined analogously to the amines. An arylamine or an aromatic amine in the sense of this invention is taken to mean a compound which contains three substituted or unsubstituted aromatic or heteroaromatic ring systems bonded directly to the nitrogen. At least one of these aromatic or heteroaromatic ring systems is preferably a condensed ring system, preferably having at least 14 aromatic ring atoms. Preferred examples thereof are aromatic anthracenamines, aromatic anthracenediamines, aromatic pyrenamines, aromatic pyrenediamines, aromatic chrysenamines or aromatic chrysenediamines. An aromatic anthracenamine is taken to mean a compound in which one diarylamino group is bonded directly to an anthracene group, preferably in the 9-position. An aromatic anthracenediamine is taken to mean a compound in which two diarylamino groups are bonded directly to an anthracene group, preferably in the 2,6- or 9,10-position. Aromatic pyrenamines, pyrenediamines, chrysenamines and chrysenediamines are defined analogously thereto, where the diarylamino groups on the pyrene are preferably bonded in the 1-position or in the 1,6-position.

Further preferred fluorescent emitter compounds are selected from indenofluorenamines or indenofluorenediamines, for example in accordance with WO 2006/122630, benzoindenofluorenamines or benzoindenofluorenediamines, for example in accordance with WO 2008/006449, and dibenzoindenofluorenamines or dibenzoindenofluorenediamines, for example in accordance with WO 2007/140847.

Further preferred fluorescent emitter compounds are selected from derivatives of naphthalene, anthracene, tetracene, benzanthracene, benzophenanthrene (DE 10 2009 005746.3), fluorene, fluoranthene, periflanthene, indenoperylene, phenanthrene, perylene (US 2007/0252517 A1), pyrene, chrysene, decacyclene, coronene, tetraphenylcyclopentadiene, pentaphenylcyclopentadiene, fluorene, spirofluorene, rubrene, coumarin (U.S. Pat. No. 4,769,292, U.S. Pat. No. 6,020,078, US 2007/0252517 A1), pyran, oxazole, benzoxazole, benzothiazole, benzimidazole, pyrazine, cinnamic acid esters, diketopyrrolopyrrole, acridone and quinacridone (US 2007/0252517 A1).

Of the anthracene compounds, particular preference is given to 9,10-substituted anthracenes, such as, for example, 9,10-diphenylanthracene and 9,10-bis(phenylethynyl)anthracene. 1,4-Bis(9′-ethynylanthracenyl)benzene is also a preferred dopant. Preference is likewise given to derivatives of rubrene, coumarin, rhodamine, quinacridone, such as, for example, DMQA (═N,N′-dimethylquinacridone), dicyanomethylenepyran, such as, for example, DCM (=4-(dicyanoethylene)-6-(4-dimethylaminostyryl-2-methyl)-4H-pyran), thiopyran, polymethine, pyrylium and thiapyrylium salts, periflanthene and indenoperylene.

Blue fluorescent emitters are preferably polyaromatic compounds, such as, for example, 9,10-di(2-naphthylanthracene) and other anthracene derivatives, derivatives of tetracene, xanthene, perylene, such as, for example, 2,5,8,11-tetra-t-butylperylene, phenylene, for example 4,4′-(bis(9-ethyl-3-carbazovinylene)-1,1′-biphenyl, fluorene, fluoranthene, arylpyrenes (U.S. Ser. No. 11/097,352 filed Apr. 4, 2005), arylenevinylenes (U.S. Pat. No. 5,121,029, U.S. Pat. No. 5,130,603), bis(azinyl)imine-boron compounds (US 2007/0092753 A1), bis(azinyl)methene compounds and carbostyryl compounds.

Further preferred blue fluorescent emitters are described in C. H. Chen et al.: “Recent developments in organic electroluminescent materials” Macromol. Symp. 125, (1997) 1-48 and “Recent progress of molecular organic electroluminescent materials and devices” Mat. Sci. and Eng. R, 39 (2002), 143-222.

Further preferred blue-fluorescent emitters are the hydrocarbons disclosed in the unpublished application DE 10 2008 035413.

Examples of fluorescent emitter compounds which can be employed in accordance with the invention are given in the following overview with the compounds having the formulae (1) to (48):

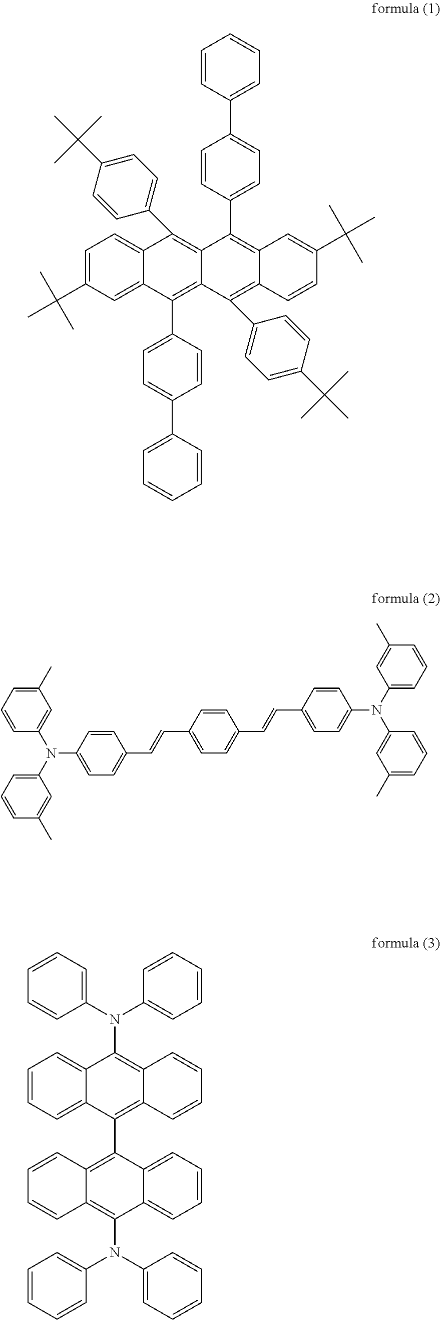

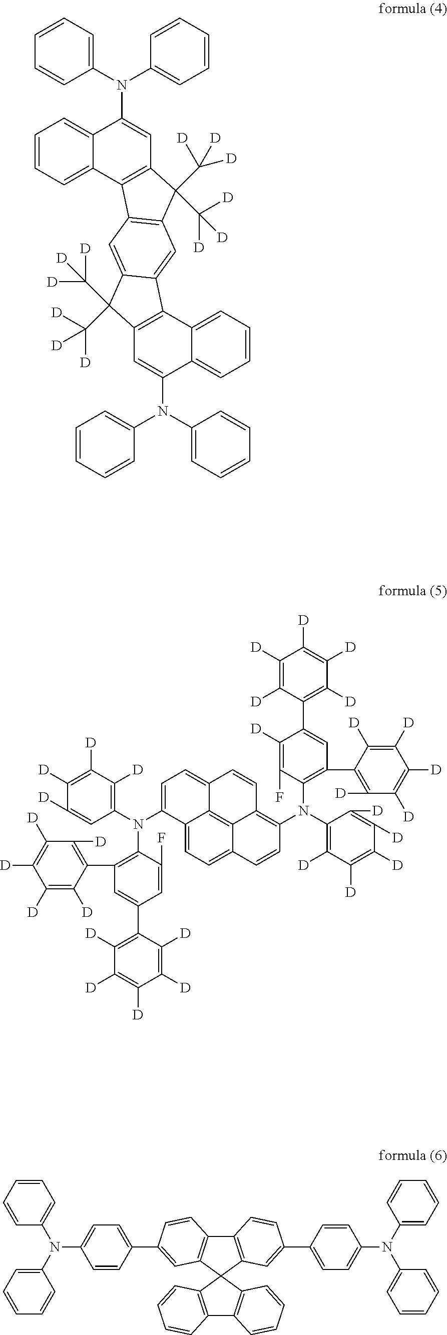

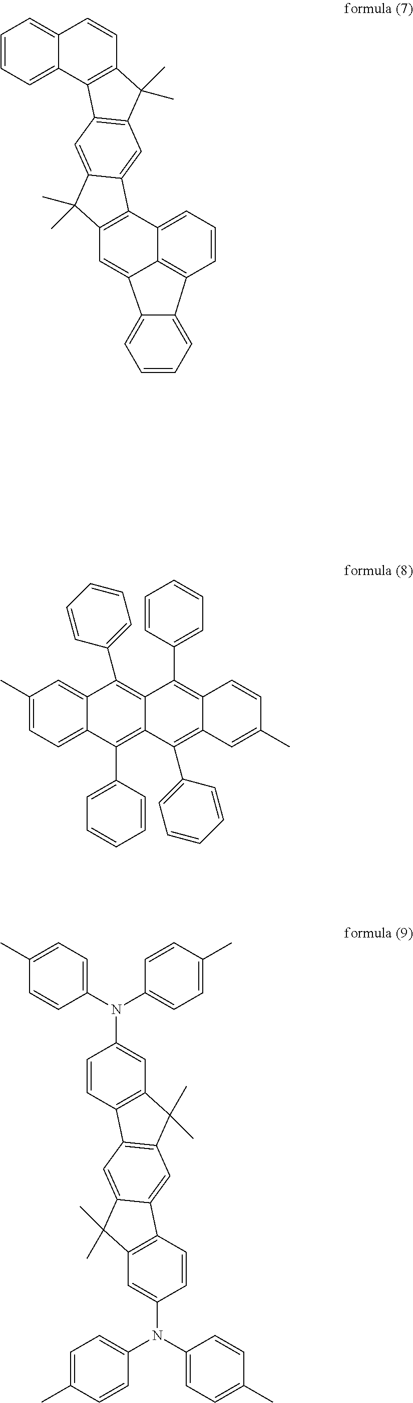

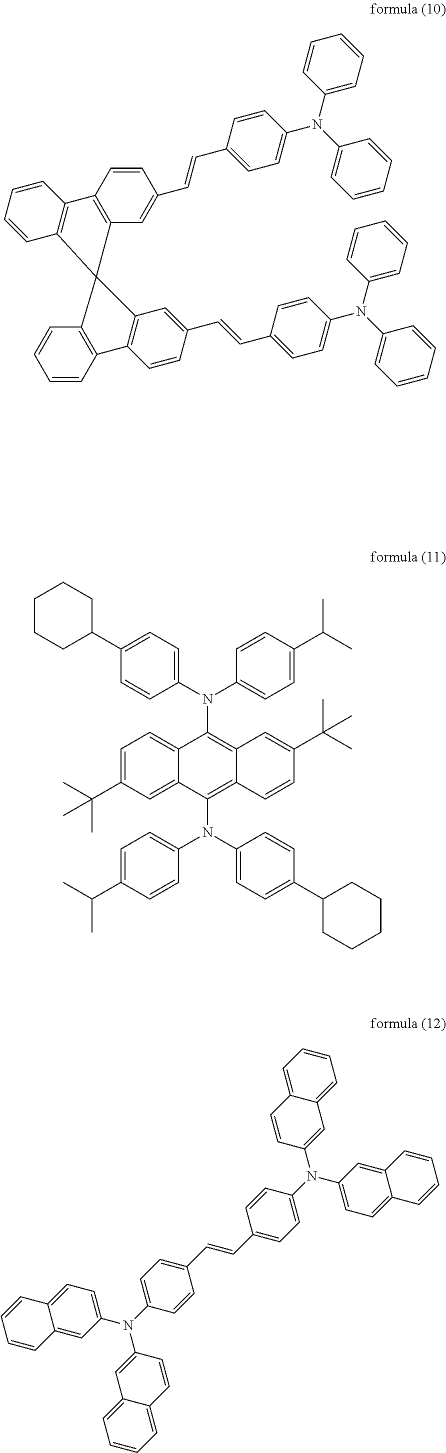 [SMILES: C(=C\c1ccc2c(c1)C1(c3ccccc3-2)c2ccccc2-c2ccc(/C=C/c3ccc(N(c4ccccc4)c4ccccc4)cc3)cc21)/c1ccc(N(c2ccccc2)c2ccccc2)cc1 ; CC(C)c1ccc(N(c2ccc(C3CCCCC3)cc2)c2c3ccc(C(C)(C)C)cc3c(N(c3ccc(C(C)C)cc3)c3ccc(C4CCCCC4)cc3)c3ccc(C(C)(C)C)cc23)cc1 ; C(=C\c1ccc(N(c2ccc3ccccc3c2)c2ccc3ccccc3c2)cc1)/c1ccc(N(c2ccc3ccccc3c2)c2ccc3ccccc3c2)cc1]

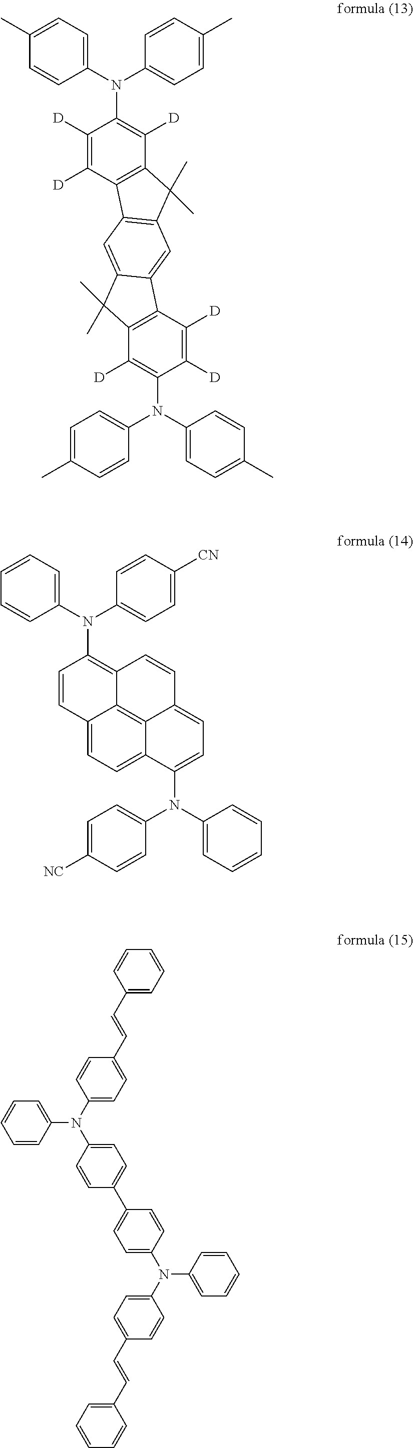

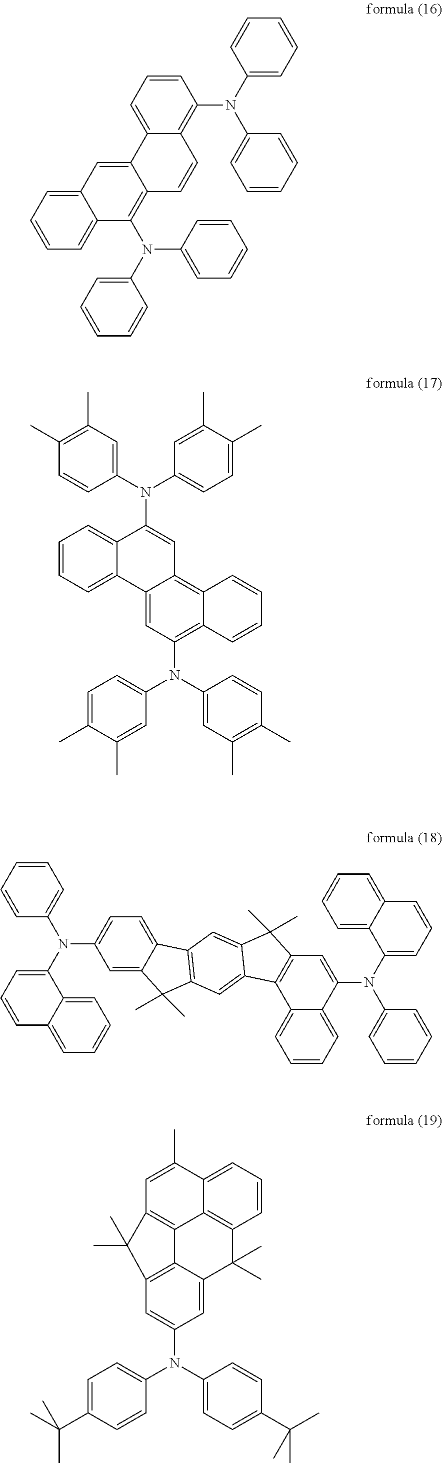

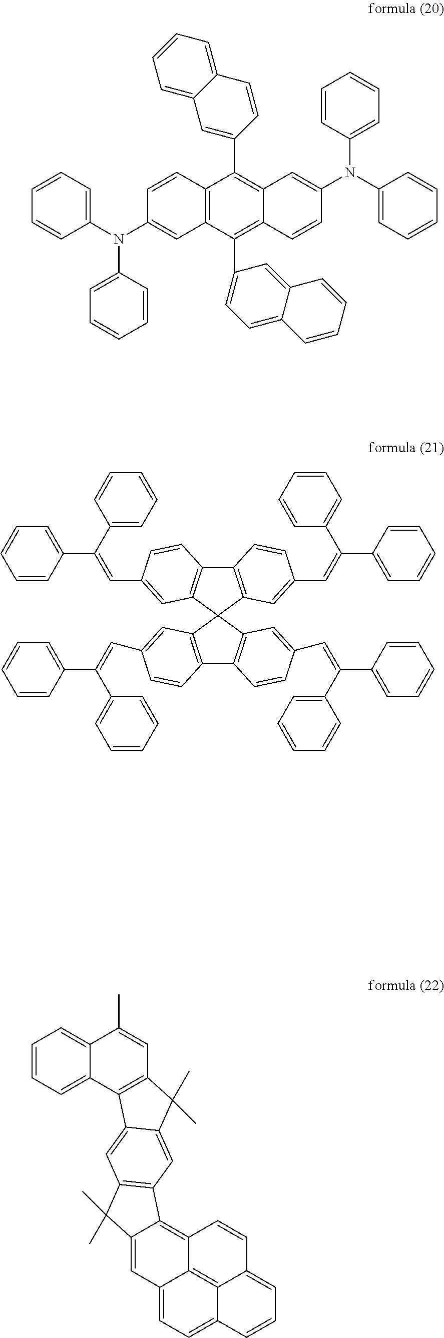

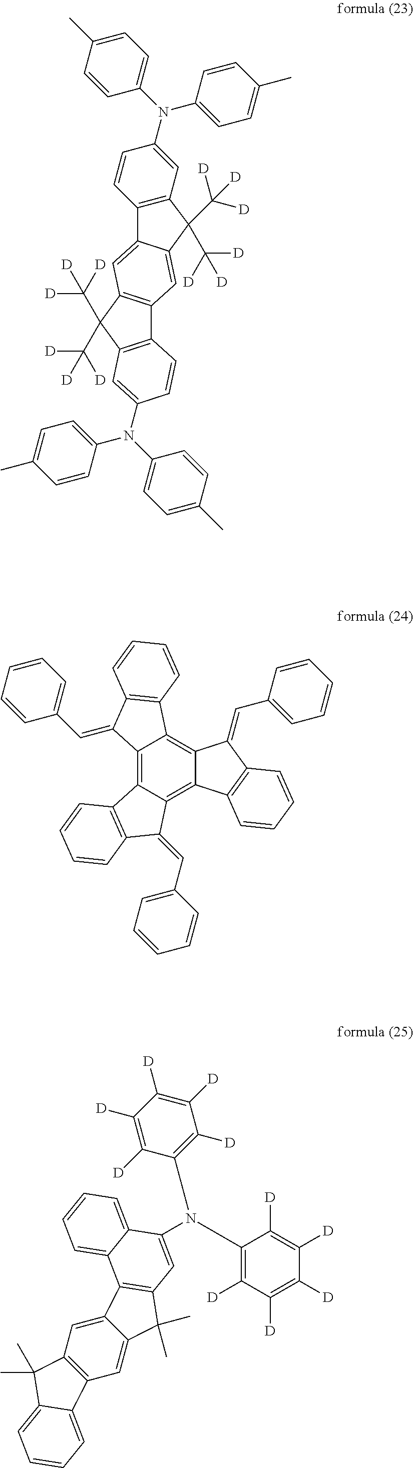

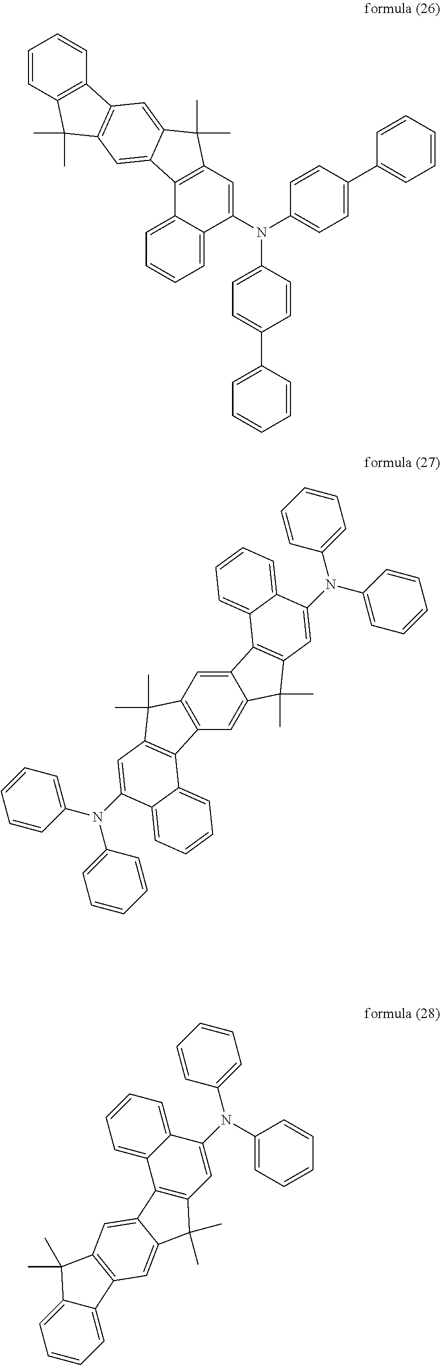 [SMILES: CC1(C)c2ccccc2-c2cc3c(cc21)-c1c(cc(N(c2ccc(-c4ccccc4)cc2)c2ccc(-c4ccccc4)cc2)c2ccccc12)C3(C)C ; CC1(C)c2cc3c(cc2-c2c1cc(N(c1ccccc1)c1ccccc1)c1ccccc21)C(C)(C)c1cc(N(c2ccccc2)c2ccccc2)c2ccccc2c1-3 ; CC1(C)c2ccccc2-c2cc3c(cc21)-c1c(cc(N(c2ccccc2)c2ccccc2)c2ccccc12)C3(C)C]

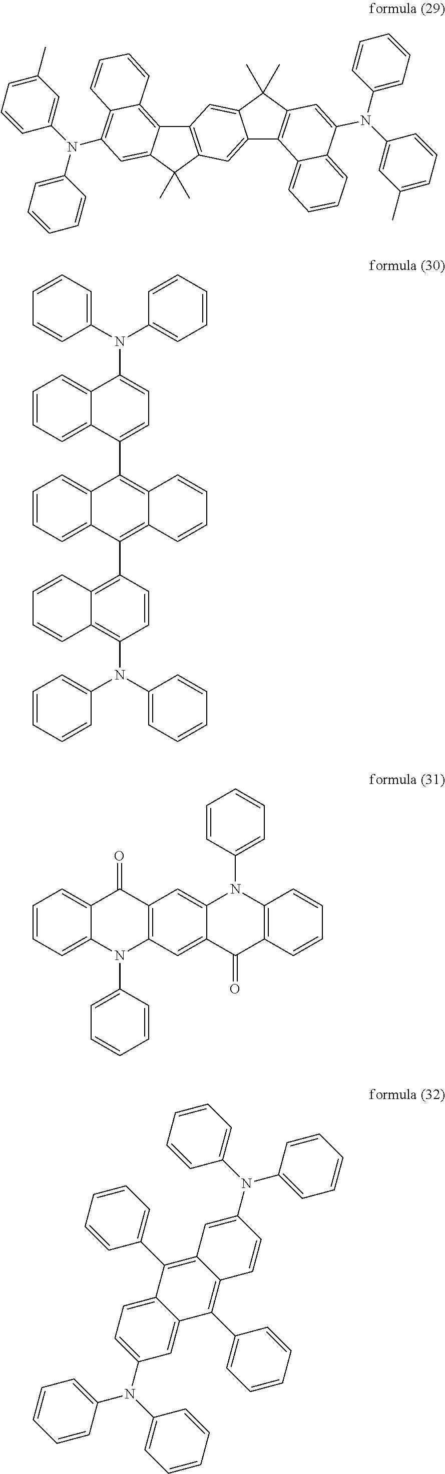

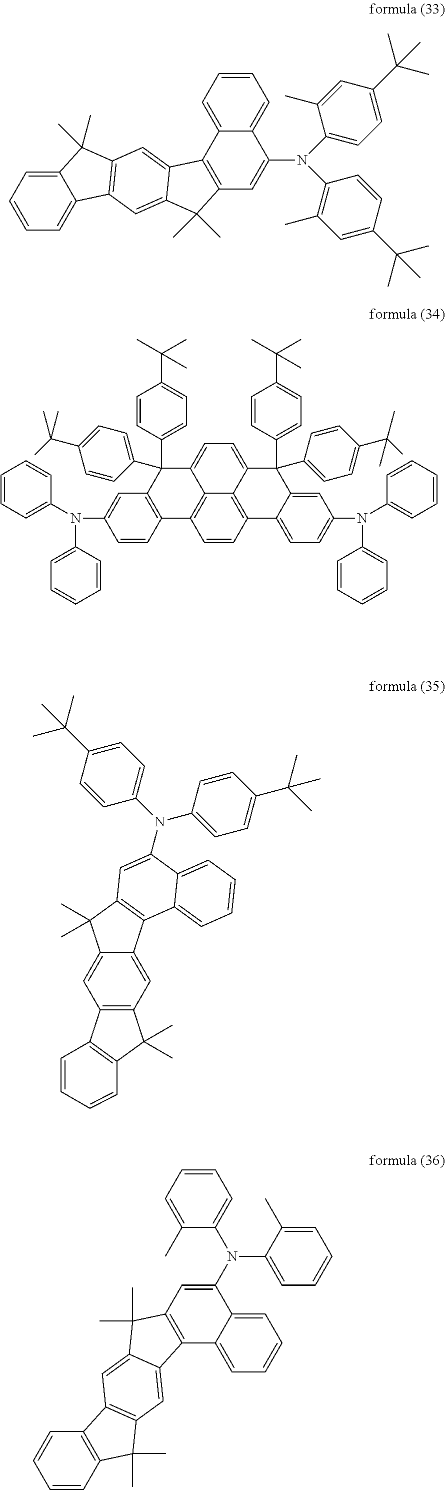

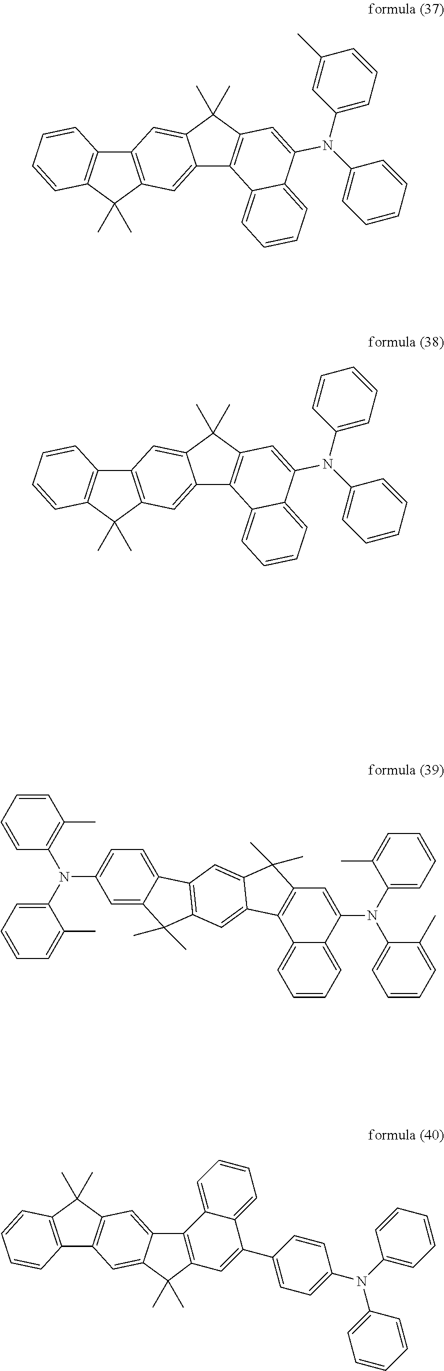 [SMILES: Cc1cccc(N(c2ccccc2)c2cc3c(c4ccccc24)-c2cc4c(cc2C3(C)C)-c2ccccc2C4(C)C)c1 ; CC1(C)c2ccccc2-c2cc3c(cc21)-c1c(cc(N(c2ccccc2)c2ccccc2)c2ccccc12)C3(C)C ; Cc1ccccc1N(c1ccc2c(c1)C(C)(C)c1cc3c(cc1-2)C(C)(C)c1cc(N(c2ccccc2C)c2ccccc2C)c2ccccc2c1-3)c1ccccc1C ; CC1(C)c2ccccc2-c2cc3c(cc21)-c1c(cc(-c2ccc(N(c4ccccc4)c4ccccc4)cc2)c2ccccc12)C3(C)C]

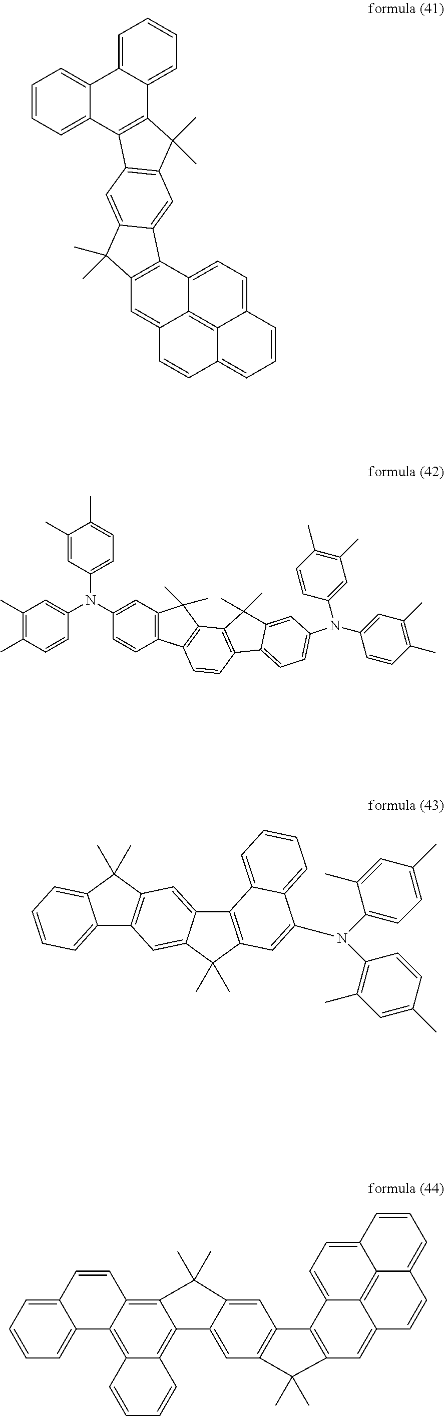

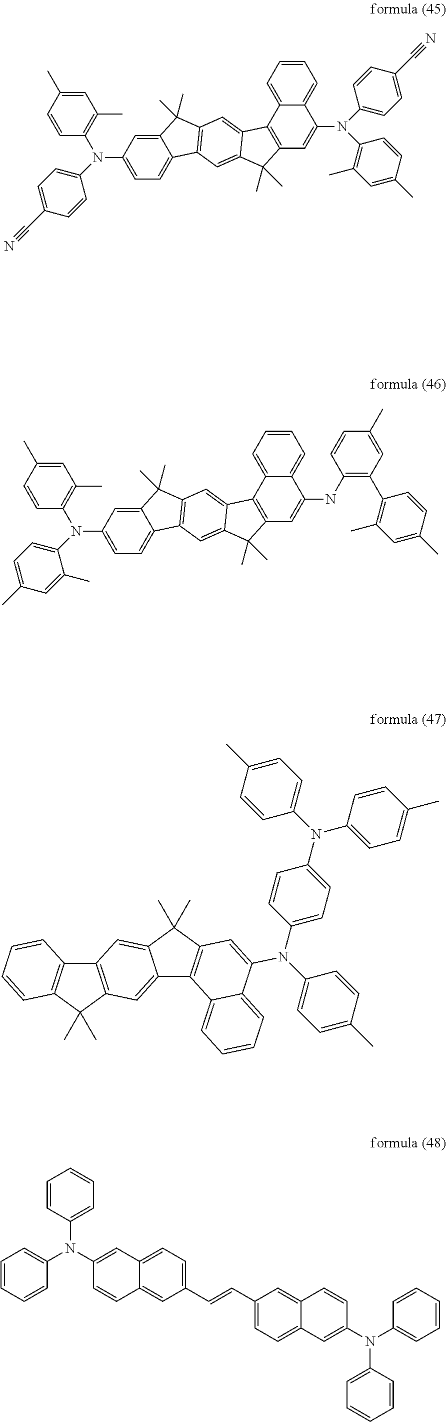

The above-mentioned fluorescent emitter compounds are preferably organic emitter compounds containing at least one charged aliphatic substituent, where the charged aliphatic substituent is preferably R₁O⁻, R₁S⁻, R₁C(O)⁻, R₁R₂R₃N⁺, R₁R₂R₃P⁺, R₁SO₃⁻, or one of the side groups mentioned below.

Suitable organic cations are selected from the group comprising ammonium, phosphonium, thiouronium and guanidinium cations, as shown in the formulae (49) to (53) or the heterocyclic cations of the formulae (54) to (81):

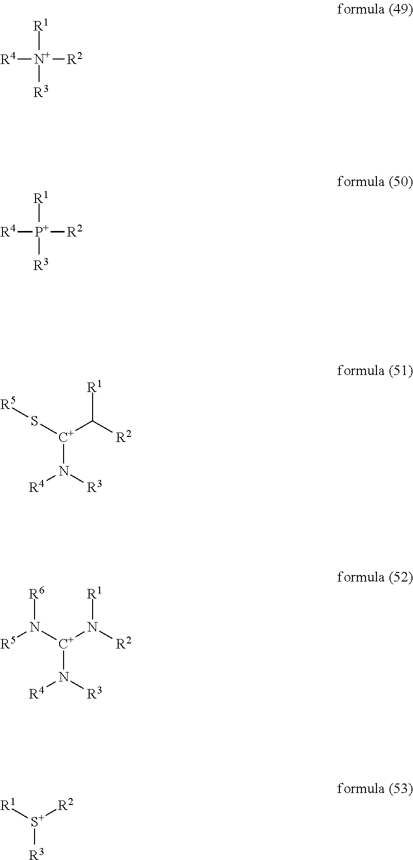

where
R¹ to R⁶ are preferably selected, independently of one another, from the group consisting of straight-chain or branched alkyl groups having 1 or 3 to 20 C atoms respectively, straight-chain or branched alkenyl groups having 2 or 3 to 20 C atoms respectively and one or more non-conjugated double bonds, straight-chain or branched alkynyl groups having 2 or 3 to 20 C atoms respectively with one or more non-conjugated triple bonds and a saturated, partially saturated or fully saturated cycloalkyl group having 3 to 7 C atoms, which may furthermore be substituted by an alkyl group having 1 to 6 C atoms, where one or more of the representatives from R¹ to R⁶ may be partially or fully substituted by a halogen, in particular by F and/or Cl, or partially substituted by —OR′, —CN, —C(O)OH, —C(O)NR′₂, —SO₂NR′₂, —SO₂OH, —SO₂X, —NO₂, where one or more non-adjacent atoms and non-α-carbon atoms from R¹ to R⁶ may be substituted by atoms or a group selected from —O—, —S—, —S(O)—, —SO₂—, —N⁺R′₂⁻, —C(O)NR′—, —SO₂NR′—, and —P(O)R′, where R′ is equal to H, a partially fluorinated or perfluorinated C₁- to C₆-alkyl group, C₃- to C₇-cycloalkyl group, an unsubstituted or substituted phenyl, and X is equal to halogen;
and in formula (49), R¹ to R⁴ may be equal to H, with the proviso that at least one representative from R¹ to R⁴ is not equal to H;
in formula (50) R¹ to R⁴ may be, independently of one another, H or NR′₂, where R′ is as defined above;
in formula (51) R¹ to R⁵ may be H;
in formula (52) R¹ to R⁶ may be, independently of one another, H, CN and
NR′₂, where R′ is as defined above.

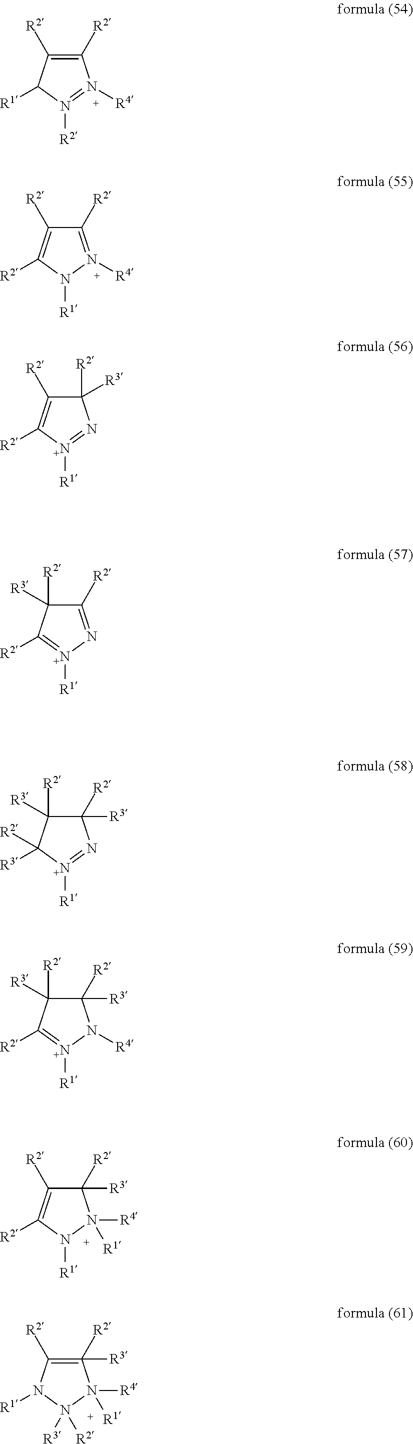

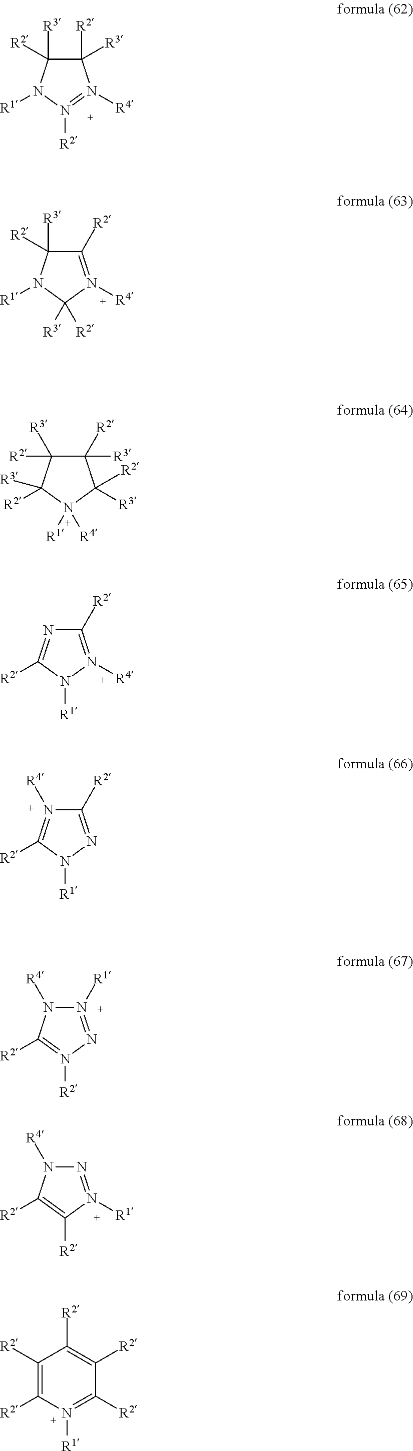

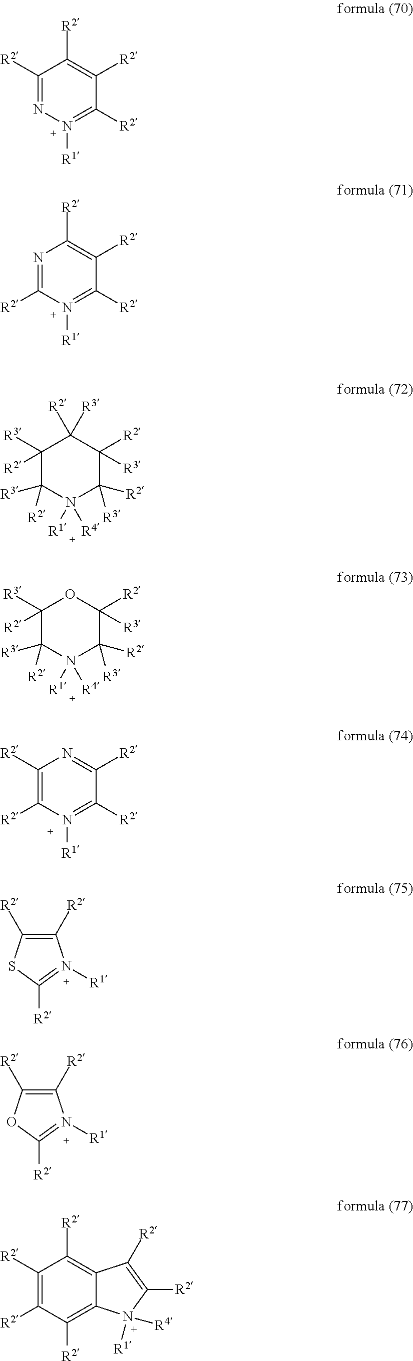

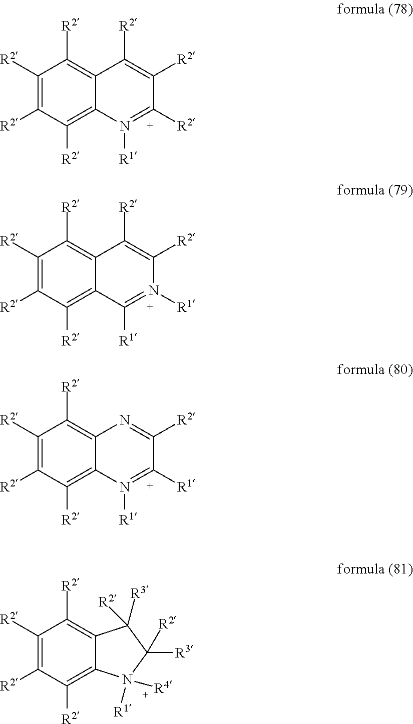

In the formulae (54) to (81), the radicals R¹′ to R⁴′ are selected, independently of one another, from the group consisting of the following: H, CN, a straight-chain or branched alkyl group having 1 or 3 to 20 C atoms respectively, a straight-chain or branched alkenyl group having 2 or 3 to 20 C atoms respectively with one or more conjugated double bond, a straight-chain or branched alkynyl group having 2 or 3 to 20 C atoms respectively with one or more non-conjugated triple bonds, a partially or fully saturated cycloalkyl group having 3 to 7 C atoms, which may be substituted by alkyl groups having 1 to 6 C atoms, a saturated or partially or fully unsaturated heteroaryl group, heteroaryl-C₁-C₆-alkyl group or alkyl-C₁-C₆-alkyl group, in which the substituents R¹′, R²′, R³′ and/or R⁴′ together may form a ring, in which one or more of the substituents from R¹′ to R⁴′ may be partially or fully substituted by halogen, preferably by F and/or Cl, and —OR′, —CN, —C(O)OH, —C(O)NR′₂, —SO₂NR′₂, —C(O)X, —SO₂OH, —SO₂X, —NO₂, in which the substituents R¹′ and R⁴′ are not both simultaneously substituted by halogen, where one or more of the carbon atoms of the substituents R¹′ and R²′ which are not adjacent to one another or bonded to a heteroatom may be substituted by a unit selected from the group consisting of —O—, —S—, —S(O)—, —SO₂—, —N⁺R′₂—, —C(O)NR′—, —SO₂NR′— and —P(O)R′—, in which R′ is equal to H, an alkyl group having 1 to 6 C atoms which is unsubstituted, partially or fully substituted by F, cycloalkyl group having 3 to 7 C atoms or unsubstituted or substituted phenyl, and X is equal to halogen.

R²′ is particularly preferably selected from —OR′, —NR′₂, —C(O)OH, —C(O)NR′₂, —SO₂NR′₂)—SO₂OH, —SO₂X and —NO₂.

Furthermore particularly preferred cationic groups contain a cation having a structure of the formula (82). These include an N,N,N-trimethylbutyl ammonium ion, N-ethyl-N,N-dimethylpropylammonium ion, N-ethyl-N,N-dimethylbutylammonium ion, N,N-dimethyl-N-propylbutylammonium ion, N-(2-methoxyethyl)-N,N-dimethylethylammonium ion, 1-ethyl-3-methyl imidazolium ion, 1-ethyl-2,3-dimethylimidazolium ion, 1-ethyl-3,4-dimethyl imidazolium ion, 1-ethyl-2,3,4-trimethylimidazolium ion, 1-ethyl-2,3,5-trimethyl imidazolium ion, N-methyl-N-propylpyrrolidinium ion, N-butyl-N-methylpyrrolidinium ion, N-sec-butyl-N-methylpyrrolidinium ion, N-(2-methoxyethyl)-N-methylpyrrolidinium ion, N-(2-ethoxyethyl)-N-methylpyrrolidinium ion, N-methyl-N-propylpiperidinium ion, N-butyl-N-methylpiperidinium ion, N-sec-butyl-N-methylpiperidinium ion, N-(2-methoxyethyl)-N-methylpiperidinium ion and N-(2-ethoxyethyl)-N-methylpiperidinium ion.

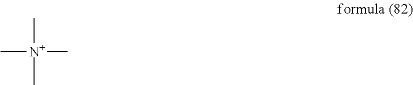

Very particular preference is given to an N-methyl-N-propylpiperidinium ion.

A particulalyr preferred ionic group is a group which imparts the salt according to the invention with good solubility in a conventional solvent, such as toluene, anisole and chloroform. This group is preferably a group selected from: methyltrioctylammonium trifluoromethanesulfonate (MATS), 1-methyl-3-octylimidazolium octylsulfate, 1-butyl-2,3-dimethylimidazolium octylsulfate, 1-octadecyl-3-methylimidazolium bis(trifluoromethylsulfonyl)-imide, 1-octadecyl-3-methylimidazolium tris(pentafluoroethyl)trifluorophosphate, 1,1-dipropylpyrrolidinium bis(trifluoromethylsulfonyl)imide, trihexyl(tetradecyl)phosphonium bis(1,2-benzenediolato(2-)-O,O′)borate and N,N,N′,N′,N′,N′-pentamethyl-N′-propylguanidinium trifluoromethane-sulfonate.

Furthermore preferred cationic groups are selected from the compounds of the following general formulae (83) to (88):

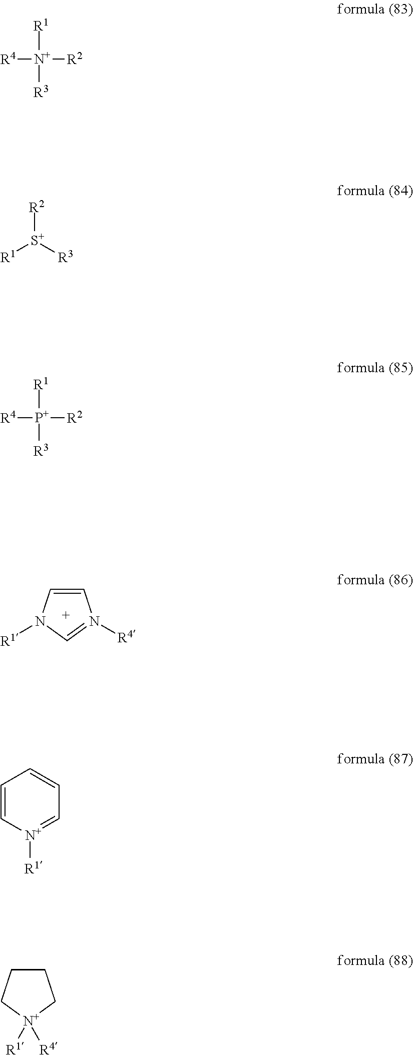

R¹ to R⁴ in the formulae (83) to (88) are defined as in the formulae (49), (53), and (50), and R^{1′} and R^{4′} as in the formulae (54), (69) and (64).

Suitable anionic side groups can be selected from [HSO₄]⁻, [SO₄]²⁻, [NO₃]⁻, [BF₄]⁻, [(R)BF3]⁻, [(R)₂BF₂]⁻, [(R)₃BF]⁻, [(R)₄B]⁻, [B(CN)₄]⁻, [PO₄]³⁻, [HPO₄]²⁻, [H₂PO₄]⁻, [alkyl-OPO₃]²⁻, [(alkyl-O)₂PO₂]⁻, [alkyl-PO₃]²⁻, [RPO₃]²⁻, [(alkyl)₂PO₂]⁻, [(R)₂PO₂]⁻, [R_{F}SO₃]⁻, [HOSO₂(CF₂)ₙSO₂O]⁻, [OSO₂(CF₂)ₙSO₂O]²⁻, [alkyl-SO₃]⁻, [HOSO₂(CH₂)ₙSO₂O]⁻, [OSO₂(CH₂)ₙSO₂O]²⁻, [alkyl-OSO₃]⁻, [alkyl-C(O)O]⁻, [HO(O)C(CH₂)ₙC(O)O]⁻, [RC(O)O]⁻, [HO(O)C(CF₂)ₙC(O)O]⁻, [O(O)C(CF₂)ₙC(O)O]²⁻, [(RSO₂)₂N]⁻, [(FSO₂)₂N]⁻, [((R)₂P(O))₂N]⁻, [(RSO₂)₃C]⁻, [(FSO₂)₃C]⁻, where n=1 to 8; R is an alkyl group, aryl group or alkylaryl group, which may optionally be fluorinated or perfluorinated.

The above-mentioned alkyl groups can be selected from straight-chain or branched alkyl groups having 1 or 3 to 20 C atoms respectively, preferably having 1 to 14 C atoms and particularly preferably 1 to 4 C atoms. R_{F} preferably denotes equal to CF₃, C₂F₅, C₃F₇ or C₄F₉.

Preferred anionic groups are: PF₆⁻, [PF₃(C₂F₅)₃]⁻, [PF₃(CF₃)₃]⁻, BF₄⁻, [BF₂(CF₃)₂]⁻, [BF₂(C₂F₅)₂]⁻, [BF₃(CF₃)]⁻, [BF₃(C₂F₅)]⁻, [B(COOCOO)₂⁻(BOB⁻), CF₃SO₃⁻(Tf⁻), C₄F₉SO₃ (Nf⁻), [(CF₃SO₂)₂N]⁻ (TFSI⁻), [(C₂F₅SO₂)₂N]⁻ (BETI⁻), [(CF₃SO₂)(C₄F₉SO₂)N]⁻, [(CN)₂N]⁻ (DCA⁻), [CF₃SO₂]₃C]⁻ and [(CN)₃C]⁻.

All the charged aliphatic substituents mentioned are covalently linked to the emitter compound, preferably through one of the hydrogen atom or fluorine atoms of the substituent not being present and the bonding to the emitter compound taking place at this position. In the case where the charged side group does not contain an aliphatic unit, a substituent of the charge-carrying central atom is preferably not present and the bond to the emitter compound is formed at this position.

The polarity and the entire charge of the emitter compound can be controlled through the selection of the type and number of the substituents.

Preferred examples of the fluorescent emitter compound which carries a charged group are given in the following overview (formulae (89) to (98)):

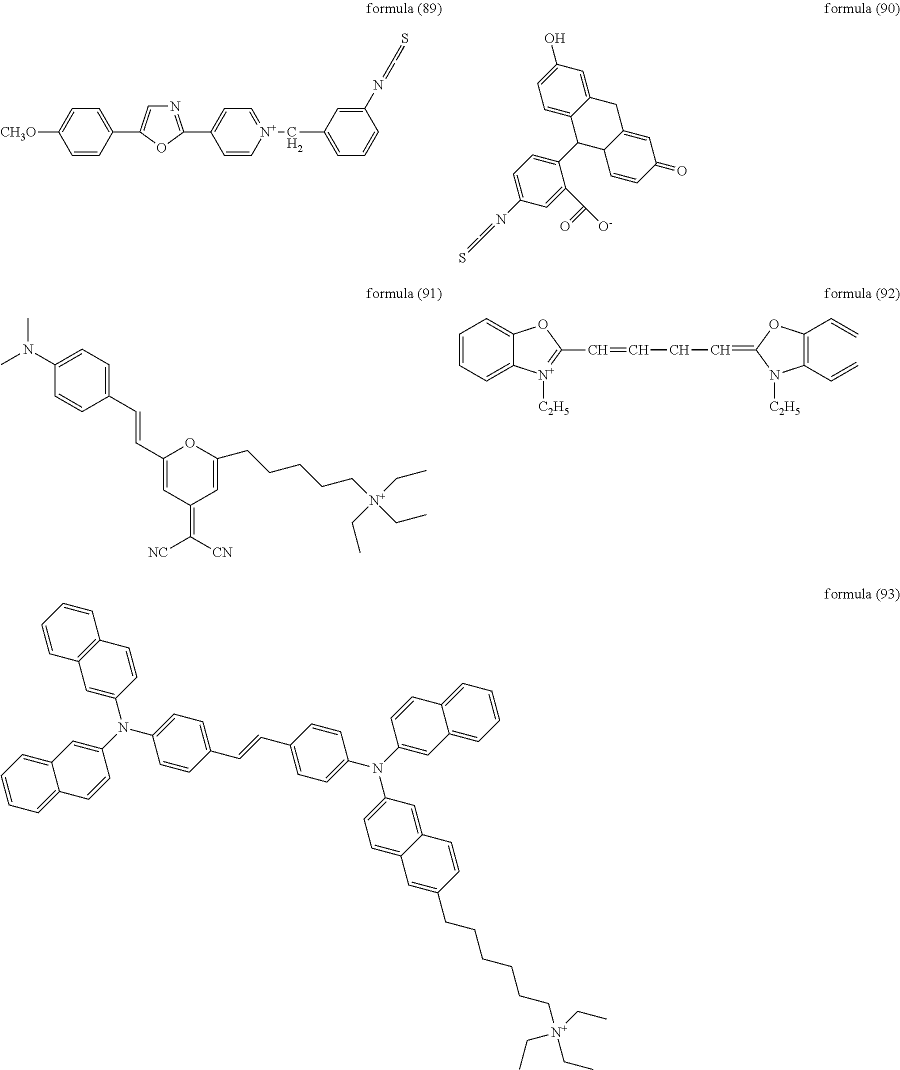

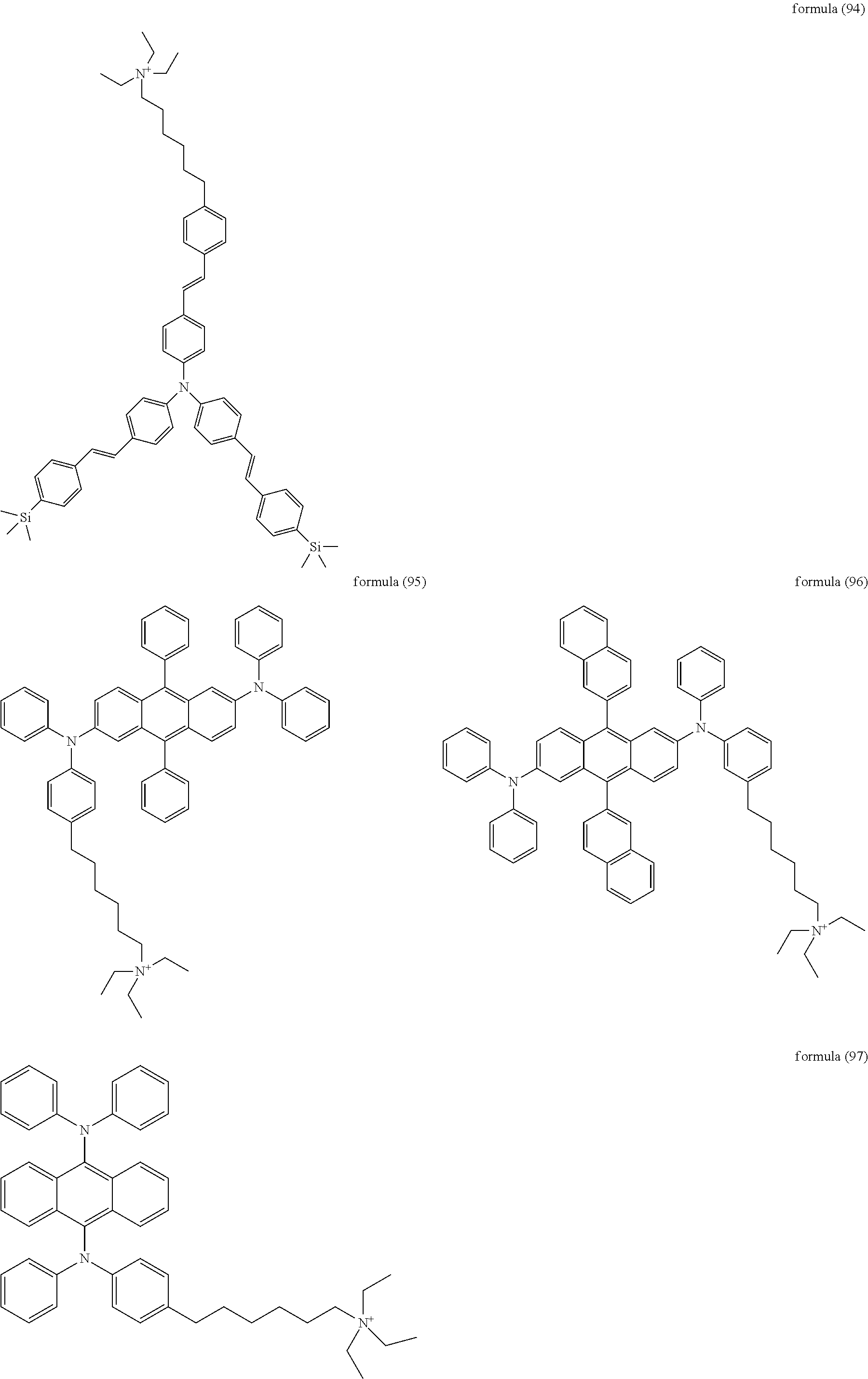

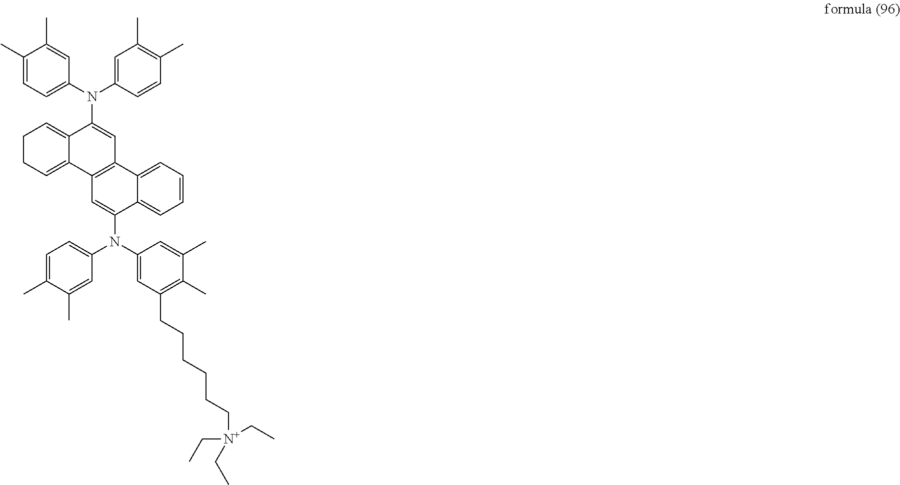 [SMILES: CC[N+](CC)(CC)CCCCCCc1cc(N(c2ccc(C)c(C)c2)c2cc3c4c(c(N(c5ccc(C)c(C)c5)c5ccc(C)c(C)c5)cc3c3ccccc23)=CCCC=4)cc(C)c1C]

Further examples of charged fluorescent emitter compounds which can be employed as ions in the salt or system according to the invention are given by the catalogue “Lambdachrome—Laser Dyes” compiled by Ulrich Brackmann (Lamda Physik); of these, the following are preferred in accordance with the invention: DASPI, DASBTI, DMETCI, DOCI, Rhodamine 110, Rhodamine 19, Rhodamine 101, DQOCI, DQTCI, DTCI, Malachit Green, Rhodamine B, DCI-2, DODCI, DTDCI, DDI, Rhodamine 19, Cresyl Violet, Nile Blue, Oxazine 4, Rhodamine 700, Pyridine 1, Oxazine 170, Oxazine 1, Oxazine 750, Pyridine 2, HIDCI, Cryptocyanine, Styryl 6, Styryl 8, Pyridine 4, Methyl-DOTCI and Styryl 11.

Due to the ionic bonding of the at least two emitter compounds or dye compounds, the salt according to the invention has excellent energy-transfer rates between the two emitter centres or between the emitter centre and the absorption centre.

The term “energy transfer” in the present invention is taken to mean a physical process in which energy is transferred from an excited dye (donor) to a second dye (acceptor) in a radiation-free manner, such as, for example, according to Förster (see T. Förster, “Zwischenmolekulare Energiewanderung and Fluoroszenz”[Intermolecular Energy Migration and Fluorescence], Ann. Physic. 437, 1948, 55) or Dexter (see D. L. Dexter, J. Chem. Phys., (1953) 21, 836).

In the present invention, one emitter compound thus preferably functions as donor, and another emitter compound or dye compound functions as acceptor in the sense of the said energy transfer.

In other words, the present invention can also be described as a system which comprises a multiplicity of different charged emitter compounds Eᵢ(nⱼ) or dye compounds,

where the symbols and indices used have the following meanings:
E(n) is an emitter compound or dye compound having the charge n;
n is a natural number which is not equal to zero; n is preferably equal to −2, −1, 1 and 2;
i is an integer which is greater than or equal to 2 and which indicates the number of non-identical emitter compounds/dye compounds occurring in the mixture; i is more preferably equal to 2 or 3;
j is an integer which is greater than or equal to 2 and which indicates the number of different charges of the emitter compounds/dye compounds occurring in the mixture; j is more preferably equal to 2;
   where the sum of the charges of all emitter compounds/dye compounds occurring in the mixture is equal to zero; and at least one of the charged emitter compounds is a fluorescent emitter compound.

In a further preferred embodiment, the dye compound is a metal-ligand coordination compound, which does not function as emitting compound, but instead as absorbing compound, and in particular as acceptor in the sense of the said energy transfer. In other words, a dye compound is not taken to mean a substance which emits light due to excitation, but instead which absorbs part of the incident light and leaves the other part “over”, which is then visible in the form of a different colour. In general, all dye-metal-ligand coordination compounds as are used in accordance with the prior art for “dye-sensitised solar cells (DSSCs)” and as are to the person skilled in the art in the area of DSSCs are suitable. In this embodiment, a dye-metal-ligand coordination compounds is preferably selected from polypyridyl complexes of transition metals, preferably ruthenium, osmium and copper. In a preferred embodiment, the dye-metal-ligand coordination compounds has the general structure ML₂(X)₂, where L is preferably 2,2′-bipyridyl-4,4′-dicarboxylic acid, M is a transition metal, preferably one from Ru, Os, Fe, V and Cu, and X is selected from halogen-, cyanide, thio-cyanate, acetylacetonate, thiocarbamate or water. Dye-metal-ligand coordination compounds of this type are disclosed, for example, in “The Journal of Physical Chemistry C 2009, 113, 2966-2973”, US 2009000658, WO 2009/107100, WO 2009/098643, U.S. Pat. No. 6,245,988, WO 2010/055471, JP 2010/084003, EP 1622178, WO 98/50393, WO 95/29924, WO 94/04497, WO 92/14741, WO 91/16719.

An example of a dye compound is:

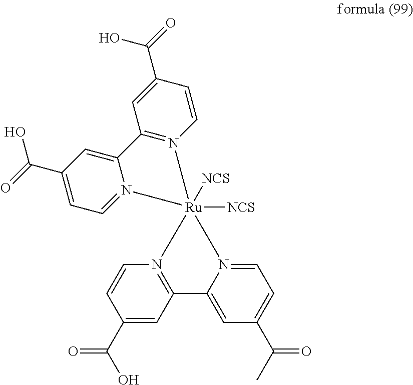

In a preferred combination, E1 is a dye compound and E2 is a fluorescent emitter compound, where the emission range of E2 overlaps with the absorption range of E1.

In a further embodiment of the present invention, all emitter compounds of thee salt or system according to the invention are preferably non-metal emitter compounds, i.e. emitter compounds which do not contain ligand coordination compounds.

In a further embodiment of the present invention, all emitter compounds of the salt or system according to the invention are preferably fluorescent emitter compounds.

In an alternative embodiment of the present invention, one of the emitter compounds of the salt or system according to the invention is a phosphorescent emitter compound. In this case, the fluorescent emitter compound preferably functions as acceptor, and the phosphorescent emitter compound as donor in the sense of the said energy transfer, so that so-called “phosphorescence sensitised fluorescence” (and has been described by Baldo, et al., in Nature (London) 403[6771], 750-753. 2000) preferably occurs, where the fluorescence properties of the fluorescent emitter compound is increased by the energy transfer. In this case, the energy levels of the two emitter compounds can also be adjusted so that extrafluorescence (and has been described by Segal, et al., in Nat. Mater. 6[5], 374-378. 2007.) preferably occurs, i.e., for example, OLEDs having higher efficiency can be obtained.

A phosphorescent emitter compound is taken to mean a compound which exhibits luminescence from an excited state having relatively high spin multiplicity, i.e. a spin state >1, such as, for example, from an excited triplet state (triplet emitter), from an MLCT mixed state or a quintet state (quintet emitter), and has a positive or negative charge. Suitable phosphorescent emitter compounds are, in particular, compounds which emit light, preferably in the visible region, on suitable excitation and in addition contain at least one atom having atomic numbers >38 and <84, particularly preferably >56 and <80. Preferred phosphorescence emitters are compounds which contain copper, molybdenum, tungsten, rhenium, ruthenium, osmium, rhodium, iridium, palladium, platinum, silver, gold or europium, in particular compounds which contain iridium, platinum or copper. Examples of the emitter compounds described above are revealed by the applications WO 00/7065, WO 01/41512, WO 02/02714, WO 02/15645, EP 1191613, EP 1191612, EP 1191614, WO 2005/033244. In general, all charged phosphorescent complexes as are used in accordance with the prior art for phosphorescent OLEDs and as are known to the person skilled in the art in the area of organic electroluminescence are suitable.

The phosphorescent emitter compound is preferably singly or doubly positively or negatively charged, i.e. represents either a cation or an anion in the salt or system according to the invention.

The phosphorescent emitter compound is preferably a metal-ligand coordination compound.

The metal-ligand coordination compound preferably contains a metal M which is a transition metal, a main-group metal or a lanthanide. If M stands for a main-group metal, it preferably stands for a metal from the third, fourth or fifth main group, in particular for tin. If M is a transition metal, it preferably stands for Ir, Ru, Os, Pt, Zn, Cu, Mo, W, Rh, Re, Ag, Au and Pd, very particularly preferably Ru and Ir. Eu is preferred as lanthanide.

M preferably stands for a transition metal, in particular for a tetracoordinated, a pentacoordinated or a hexacoordinated transition metal, particularly preferably selected from the group consisting of chromium, molybdenum, tungsten, rhenium, ruthenium, osmium, rhodium, iridium, nickel, palladium, platinum, copper, silver and gold, in particular molybdenum, tungsten, rhenium, ruthenium, osmium, iridium, platinum, copper and gold. Very particular preference is given to iridium and platinum. The metals here can be in various oxidation states. The above-mentioned metals are preferably in the oxidation states Cr(0), Cr(II), Cr(III), Cr(IV), Cr(VI), Mo(0), Mo(II), Mo(III), Mo(IV), Mo(VI), W(0), W(II), W(III), W(IV), W(VI), Re(I), Re(II), Re(III), Re(IV), Ru(II), Ru(III), Os(II), Os(III), Os(IV), Rh(I), Rh(III), Ir(I), Ir(III), Ir(IV), Ni(0), Ni(II), Ni(IV), Pd(II), Pt(II), Pt(IV), Cu(I), Cu(II), Cu(III), Ag(I), Ag(II), Au(I), Au(III) and Au(V); very particular preference is given to Mo(0), W(0), Re(I), Ru(II), Os(II), Rh(III), Ir(III), Pt(II) and Cu(I), in particular Ir(III) and Pt(II).

In a preferred embodiment of the invention, M is a tetracoordinated or hexacoordinated metal.

The ligands of the metal-ligand coordination compound are preferably mono-, bi-, tri-, tetra-, penta- or hexadentate ligands.

If M is a hexacoordinated metal, the following coordinative possibilities exist, depending on the number q of ligands:
q=2: M is coordinated to two tridentate ligands or to one tetradentate and one bidentate ligand or to one pentadentate and one monodentate ligand;
q=3: M is coordinated to three bidentate ligands or to one tridentate, one bidentate and one monodentate ligand or to one tetradentate and two monodentate ligands;
q=4: M is coordinated to two bidentate and two monodentate ligands or one tridentate and three monodentate ligands;
q=5: M is coordinated to one bidentate and four monodentate ligands;
q=6: M is coordinated to 6 monodentate ligands.

It is particularly preferred if M is a hexacoordinated metal, q=4 and two ligands are bidentate ligands and two ligands are monodentate ligands.

If M is a tetracoordinated metal, the denticity of the ligands is as follows, depending on q, which indicates the number of ligands:
q=2: M is coordinated to two bidentate ligands or to one tridentate and one monodentate ligand;
q=3: M is coordinated to one bidentate and two monodentate ligands;
q=4: M is coordinated to four monodentate ligands.

It is particularly preferred if M is a tetracoordinated metal, q=3 and one ligand is a bidentate ligand and two ligands are monodentate ligands.

The ligands of the metal-ligand coordination compound are preferably neutral, monoanionic, dianionic or trianionic ligands, particularly preferably neutral or monoanionic ligands. They can be monodentate, bidentate, tridentate, tetradentate pentadentate or hexadentate, and are preferably bidentate, i.e. preferably have two coordination sites.

It is furthermore preferred in accordance with the invention if in each case at least one ligand of the metal-ligand coordination compound is a bidentate ligand.

Preferred neutral, monodentate ligands of the metal-ligand coordination compound are selected from carbon monoxide, nitrogen monoxide, alkylcyanides, such as, for example, acetonitrile, arylcyanides, such as, for example, benzonitrile, alkylisocyanides, such as, for example, methylisonitrile, arylisocyanides, such as, for example, benzoisonitrile, amines, such as, for example, trimethylamine, triethylamine, morpholine, phosphines, in particular halophosphines, trialkylphosphines, triarylphosphines or alkylarylphosphines, such as, for example, trifluorophosphine, trimethylphosphine, tricyclohexylphosphine, tri-tert-butylphosphine, triphenylphosphine, tris(pentafluorophenyl)phosphine, phosphites, such as, for example, trimethyl phosphite, triethyl phosphite, arsines, such as, for example, trifluoroarsine, trimethylarsine, tricyclohexylarsine, tri-tert-butylarsine, triphenylarsine, tris(pentafluorophenyl)arsine, stibines, such as, for example, trifluorostibine, trimethylstibine, tricyclohexylstibine, tri-tert-butylstibine, triphenylstibine, tris(pentafluorophenyl)stibine, nitrogen-containing heterocycles, such as, for example, pyridine, pyridazine, pyrazine, pyrimidine, triazine, and carbenes, in particular Arduengo carbenes.

Preferred monoanionic, monodentate ligands of the metal-ligand coordination compound are selected from hydride, deuteride, the halides F⁻, Cl⁻, Br⁻and I⁻, alkylacetylides, such as, for example, methyl-C≡C⁻, tert-butyl-C≡C⁻, arylacetylides, such as, for example, phenyl-C≡C⁻, cyanide, cyanate, isocyanate, thiocyanate, isothiocyanate, aliphatic or aromatic alcoholates, such as, for example, methanolate, ethanolate, propanolate, isopropanolate, tert-butylate, phenolate, aliphatic or aromatic thio-alcoholates, such as, for example, methanethiolate, ethanethiolate, propanethiolate, isopropanethiolate, tert-thiobutylate, thiophenolate, amides, such as, for example, dimethylamide, diethylamide, diisopropylamide, morpholide, carboxylates, such as, for example, acetate, trifluoroacetate, propionate, benzoate, aryl groups, such as, for example, phenyl, naphthyl, and anionic, nitrogen-containing heterocycles, such as pyrrolide, imidazolide, pyrazolide. The alkyl groups in these groups are preferably a C₁-C₄₀-alkyl, particularly preferably C₁-C₁₀-alkyl, very particularly preferably C₁-C₄-alkyl. An aryl group is also taken to mean heteroaryl groups. These groups are defined as below.

Preferred di- or trianionic ligands of the metal-ligand coordination compound are O²⁻, S²⁻, carbides, which result in coordination in the form R—C≡M, nitrenes, which result in coordination in the form R—N=M, where R generally stands for a substituent, and N³⁻.

Preferred neutral or mono- or dianionic bidentate or polydentate ligands of the metal-ligand coordination compound are selected from diamines, such as, for example, ethylenediamine, N,N,N′,N′-tetramethylethylenediamine, propylenediamine, N,N,N′,N′-tetramethylpropylenediamine, cis- or trans-diaminocyclohexane, cis- or trans-N,N,N′,N′-tetramethyldiaminocyclohexane, imines, such as, for example, 2[1-(phenylimino)ethyl]pyridine, 2[1-(2-methylphenylimino)ethyl]pyridine, 2[1-(2,6-di-iso-propylphenylimino)-ethyl]pyridine, 2[1-(methylimino)ethyl]pyridine, 2[1-(ethylimino)ethyl]pyridine, 2[1-(iso-propylimino)ethyl]pyridine, 2[1-(tert-butylimino)ethyl]pyridine, diimines, such as, for example, 1,2-bis(methylimino)ethane, 1,2-bis(ethylimino)ethane, 1,2-bis(iso-propylimino)ethane, 1,2-bis(tert-butylimino)-ethane, 2,3-bis(methylimino)butane, 2,3-bis(ethylimino)butane, 2,3-bis(iso-propylimino)butane, 2,3-bis(tert-butylimino)butane, 1,2-bis(phenylimino)-ethane, 1,2-bis(2-methylphenylimino)ethane, 1,2-bis(2,6-di-iso-propylphenylimino)ethane, 1,2-bis(2,6-di-tert-butylphenylimino)ethane, 2,3-bis-(phenylimino)butane, 2,3-bis(2-methylphenylimino)butane, 2,3-bis(2,6-di-iso-propylphenylimino)butane, 2,3-bis(2,6-di-tert-butylphenylimino)butane, heterocycles containing two nitrogen atoms, such as, for example, 2,2′-bipyridine, o-phenanthroline, diphosphines, such as, for example, bis-(diphenylphosphino)methane, bis(diphenylphosphino)ethane, bis(diphenylphosphino)propane, bis(diphenylphosphino)butane, bis(dimethylphosphino)methane, bis(dimethylphosphino)ethane, bis(dimethylphosphino)-propane, bis(diethylphosphino)methane, bis(diethylphosphino)ethane, bis(diethylphosphino)propane, bis(di-tert-butylphosphino)methane, bis(di-tert-butylphosphino)ethane, bis(tert-butylphosphino)propane, 1,3-diketonates derived from 1,3-diketones, such as, for example, acetylacetone, benzoylacetone, 1,5-diphenylacetylacetone, dibenzoylmethane, bis(1,1,1-trifluoroacetyl)methane, 3-ketonates derived from 3-ketoesters, such as, for example, ethyl acetoacetate, carboxylates derived from aminocarboxylic acids, such as, for example, pyridine-2-carboxylic acid, quinoline-2-carboxylic acid, glycine, N,N-dimethylglycine, alanine, N,N-dimethylaminoalanine, salicyliminates derived from salicylimines, such as, for example, methylsalicylimine, ethylsalicylimine, phenylsalicylimine, dialcoholates derived from dialcohols, such as, for example, ethylene glycol, 1,3-propylene glycol, and dithiolates derived from dithiols, such as, for example, 1,2-ethylenedithiol, 1,3-propylenedithiol.

Preferred tridentate ligands are borates of nitrogen-containing hetero-cycles, such as, for example, tetrakis(1-imidazolyl) borate and tetrakis-(1-pyrazolyl) borate.

Preference is furthermore given to bidentate monoanionic ligands of the metal-ligand coordination compound which, with the metal, have a cyclometallated five-membered ring or six-membered ring having at least one metal-carbon bond, in particular a cyclometallated five-membered ring. These are, in particular, ligands as generally used in the area of phosphorescent metal-ligand coordination compounds for organic electroluminescent devices, i.e. ligands of the phenylpyridine, naphthylpyridine, phenylquinoline, phenylisoquinoline, etc., type, each of which may be substituted by one or more radicals R. A multiplicity of ligands of this type is known to the person skilled in the art in the area of phosphorescent electroluminescent devices, and he will be able to select further ligands of this type. In general, the combination of two groups, as represented by the following formulae (L-1) to (L-28), is particularly suitable for this purpose, where one group is bonded via a neutral nitrogen atom or a carbene atom and the other group is bonded to the metal via a negatively charged carbon atom or a negatively charged nitrogen atom. The ligand of the metal-ligand coordination compound can then be formed from the groups of the formulae (100) to (127) through these groups bonding to one another, in each case at the position denoted by #. The position at which the groups coordinate to the metal are denoted by *.

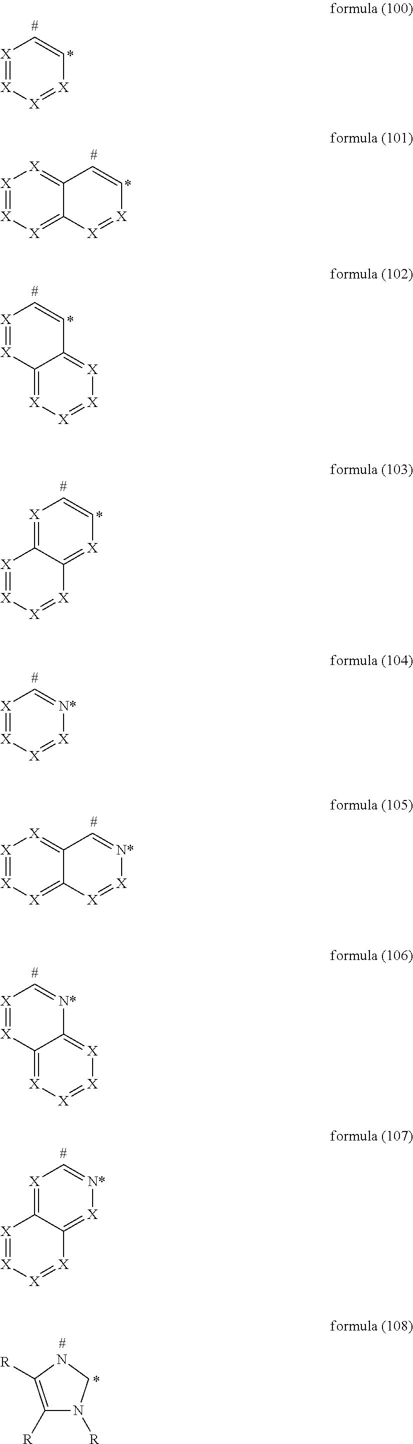

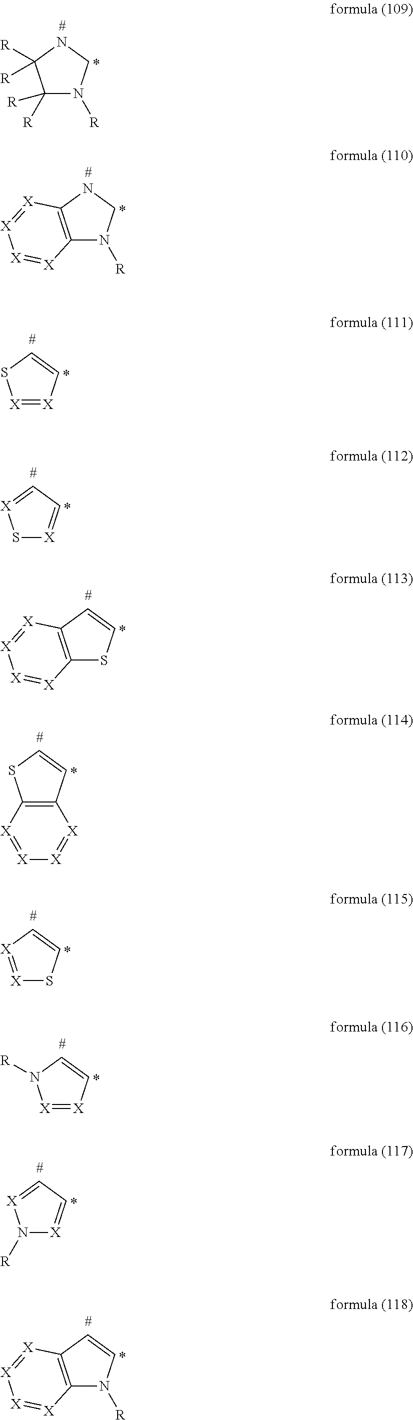

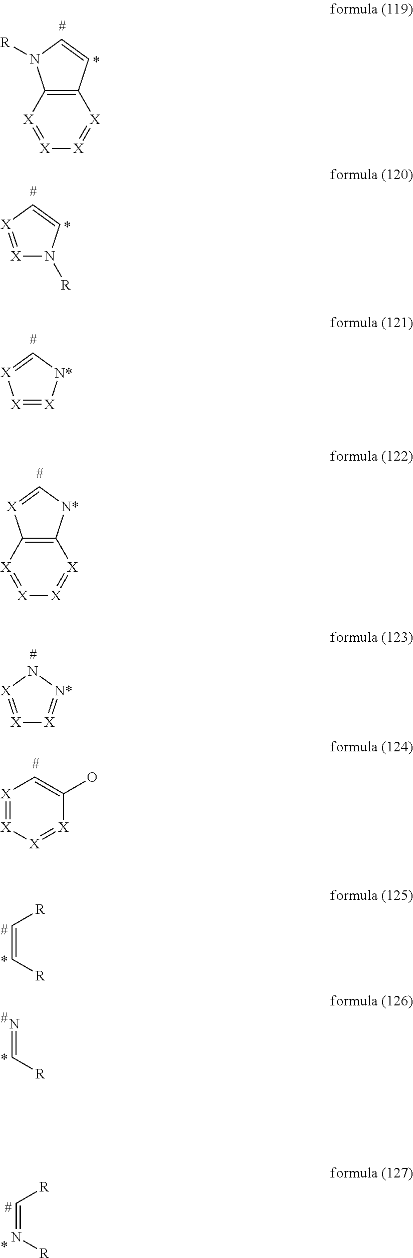

R here is selected on each occurrence, identically or differently, from the group consisting of alkyl, alkylsilyl, silyl, arylsilyl, alkoxyalkyl, arylalkoxyalkyl, alkylthioalkyl, phosphine, phosphine oxide, sulfone, alkyl sulfone, sulfoxide and alkyl sulfoxide, where the alkyl group in each case has, independently of one another, 1 to 12 C atoms and where one or more H atoms may be replaced by F, Cl, Br, I, alkyl or cycloalkyl, where one or more CH₂ may be replaced by a heteroatom, such as NH, O or S, or an aromatic or heteroaromatic hydrocarbon radical having 5 to 40 aromatic ring atoms. X stands for N or CH. Particularly preferably a maximum of three symbols X in each group stand for N, particularly preferably a maximum of two symbols X in each group stand for N, very particularly preferably a maximum of one symbol X in each group stands for N. Especially preferably, all symbols X stand for CH.

Likewise preferred ligands are η⁵-cyclopentadienyl, η⁵-pentamethylcyclopentadienyl, η⁶-benzene and η⁷-cycloheptatrienyl, each of which may be substituted by one or more radicals R.

Likewise preferred ligands are 1,3,5-cis-cyclohexane derivatives, in particular of the formula (128), 1,1,1-tri(methylene)methane derivatives, in particular of the formula (129), and 1,1,1-trisubstituted methanes, in particular of the formulae (130) and (131),

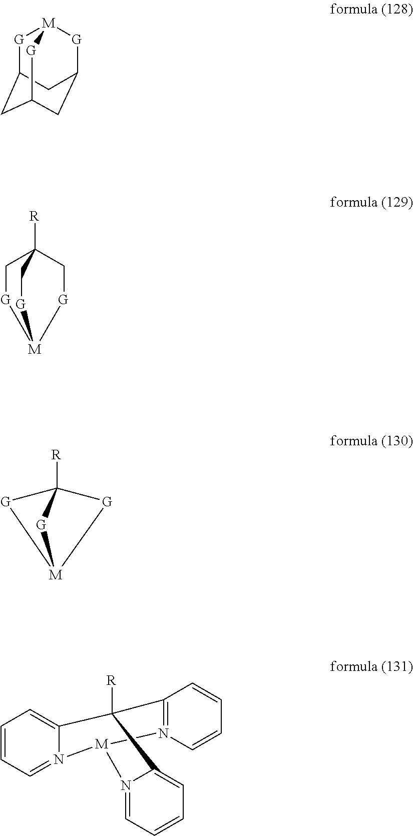

where the coordination to the metal M is depicted in each of the formulae, R has the above-mentioned meaning and G stands, identically or differently on each occurrence, for O⁻, S⁻, COO⁻, P(R)₂ or N(R)₂.

The metal-ligand coordination compound can be an anionic or cationic coordination compound, i.e. the valence of the metal M and the valence of the ligands of the metal-ligand coordination compound is selected so that the charge within each coordination compound is not compensated.

Examples of phosphorescent emitter compounds are given in the overview (formulae (132) to (188)):

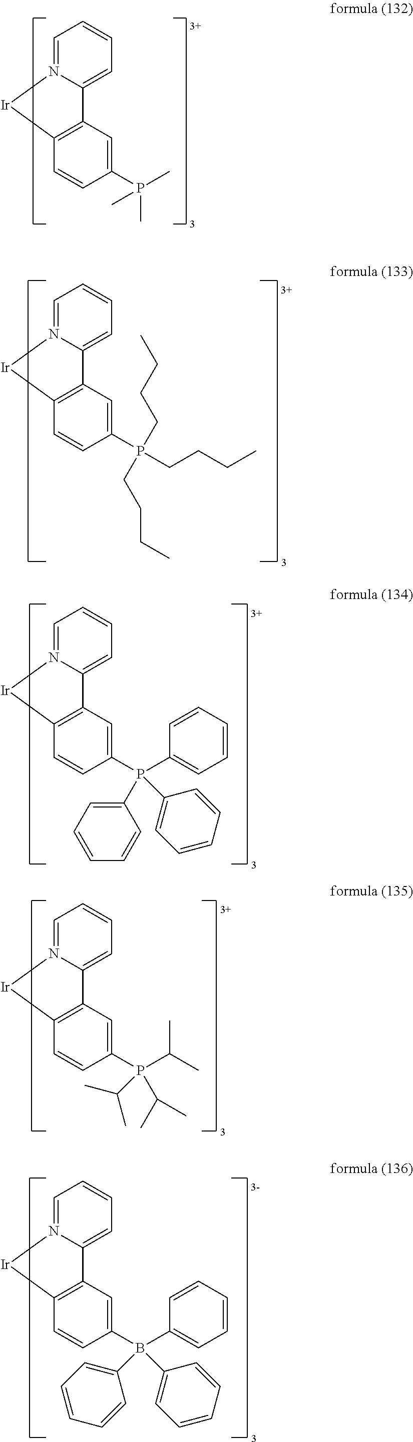

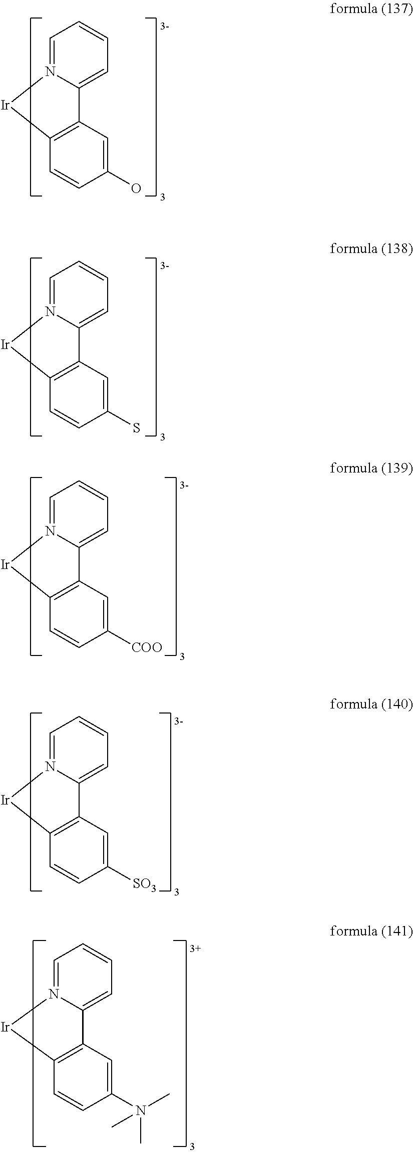

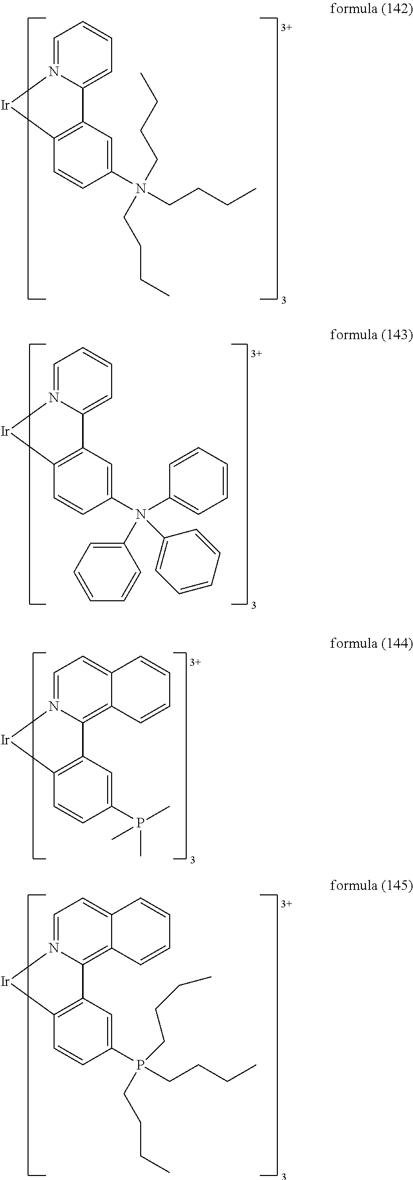

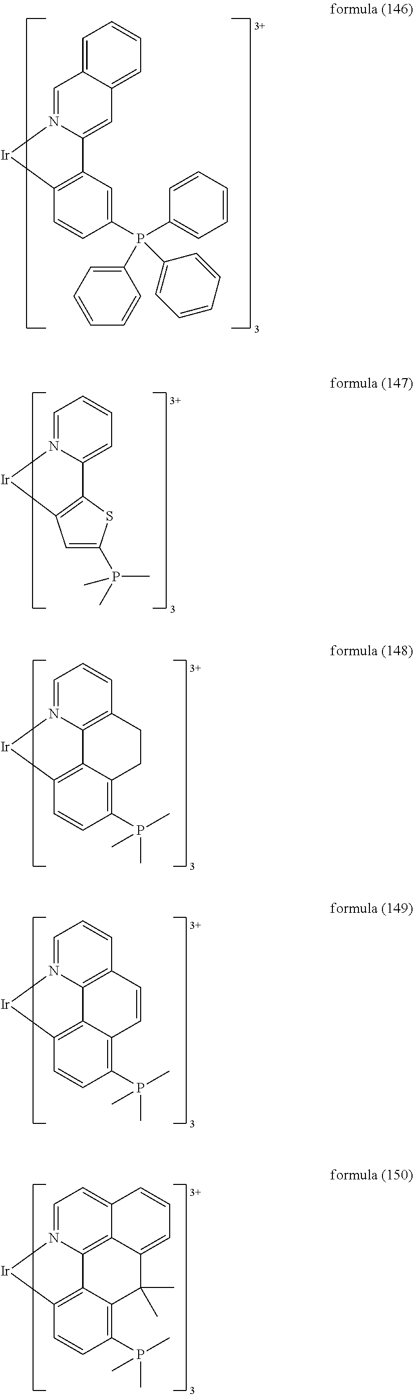

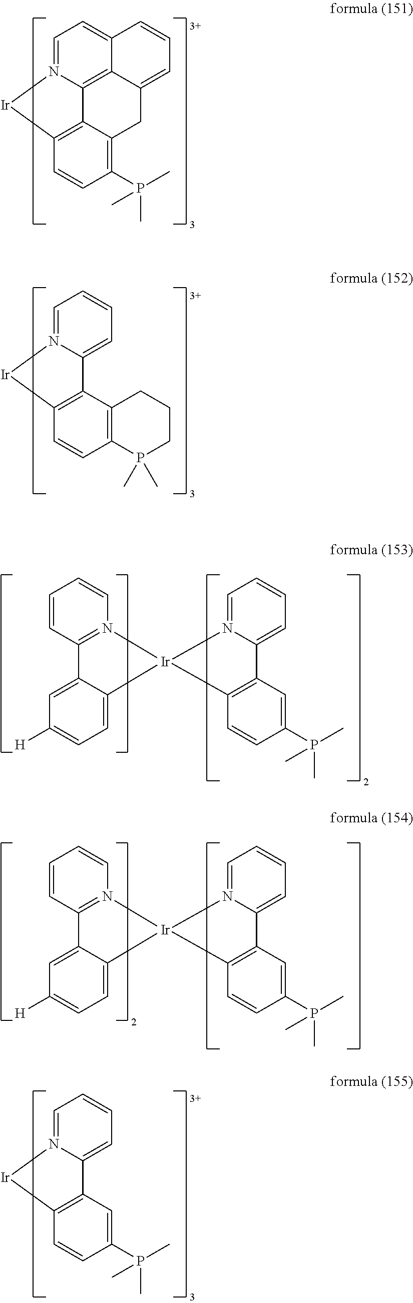

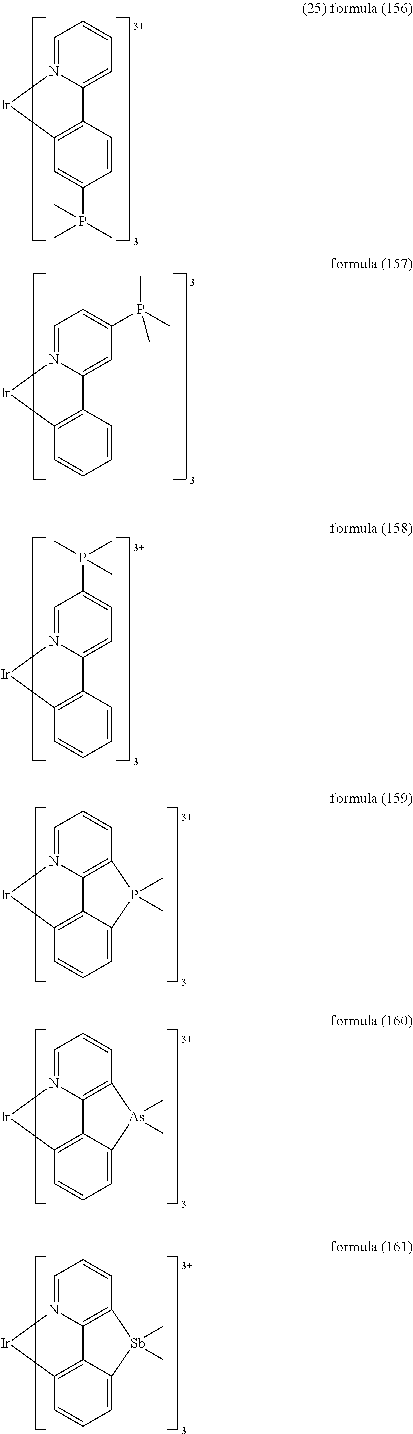

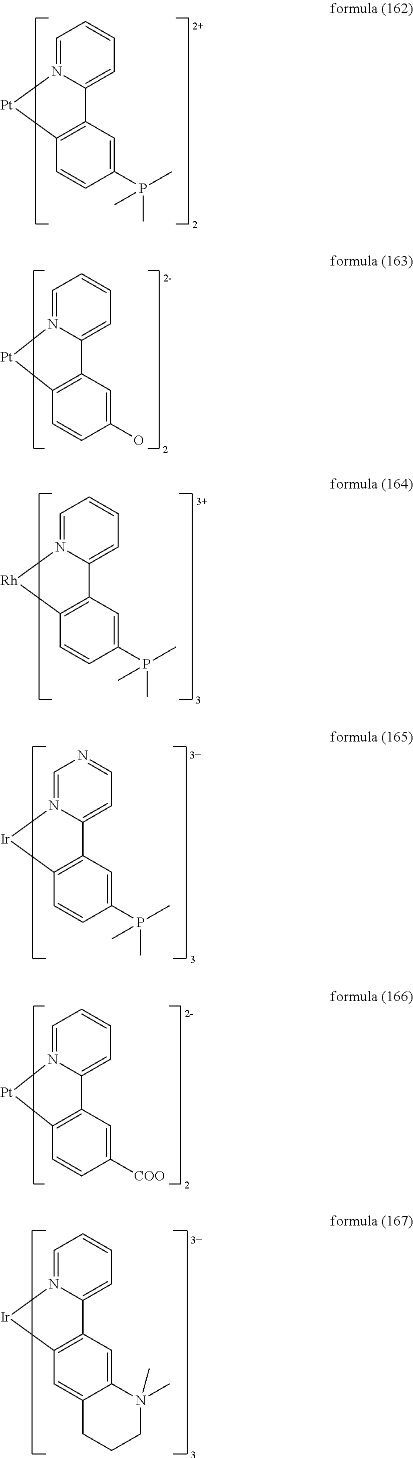

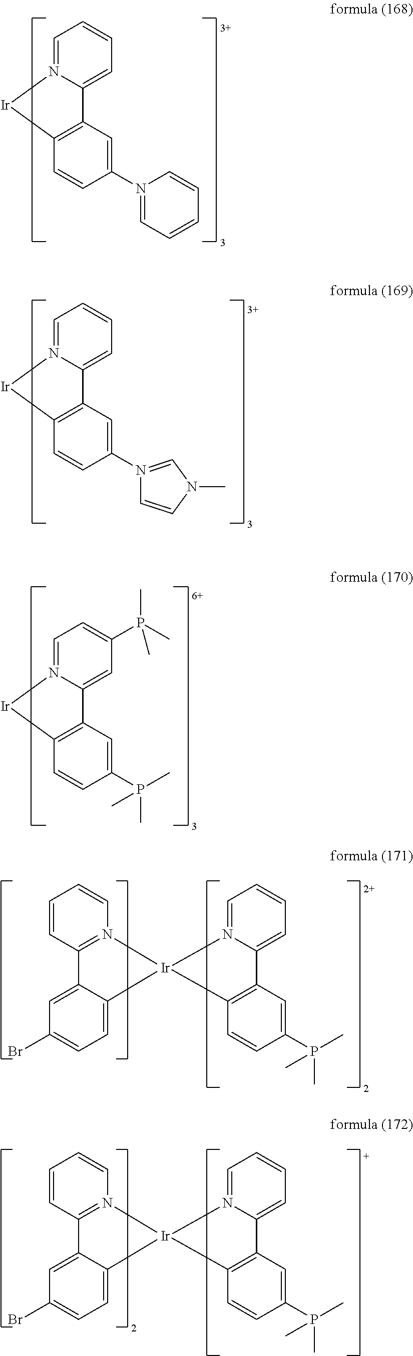

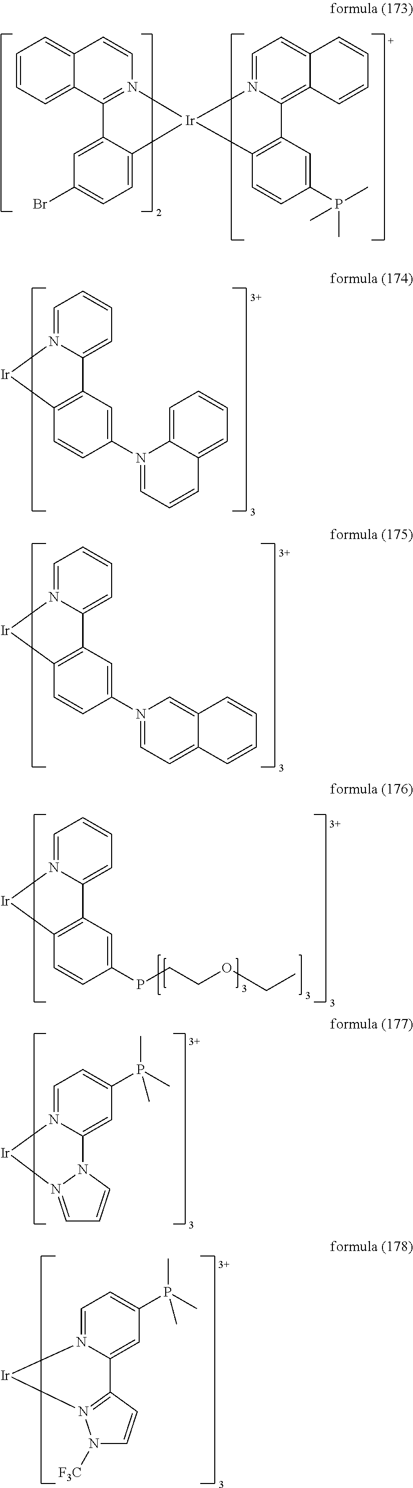

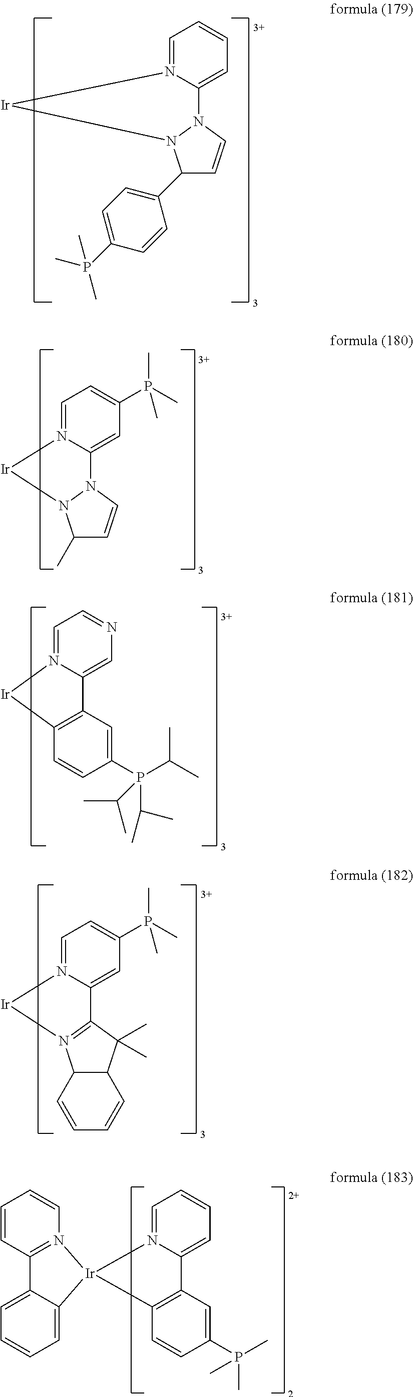

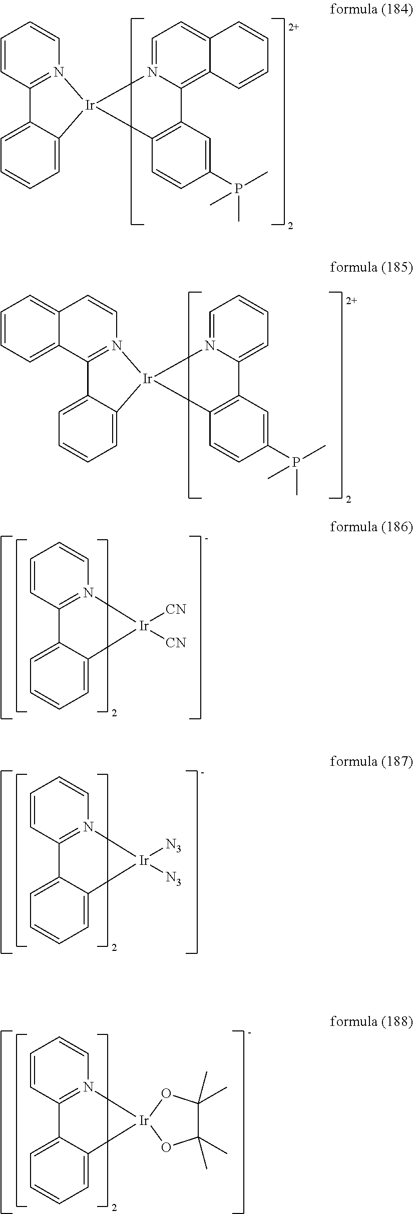

It is furthermore preferred for the salt or system according to the invention to be in the form [E₁]^{+m}[E₂]^{-m}, where [E₁]^{+m} is an emitter compound having the charge +m and [E₂]^{-m} is an emitter compound having the charge −m, where m is equal to 1 or 2. In this case, m is particuarly preferably equal to 1.

[E¹]^{-m} here can be a phosphorescent emitter compound T^{-m} and [E₂]^{+m} can be a fluorescent emitter compound S^{+m}, or [E₁]^{+m} and [E₂]^{-m} are fluorescent emitter compounds S₁^{+m} and S₂^{-m}. Here too, it is preferred for m to be equal to 1.

It is furthermore preferred for the emitter compounds [E₁]^{-m} and [E₂]^{+m} to have an energy “offset” structure of type II, in which the HOMO (highest occupied molecular orbital) of a first emitter compound is below the HOMO of the second emitter compound and the LUMO (lowest unoccupied molecular orbital) of the first emitter compound is below the LUMO of the second emitter compound, but above the HOMO of the second emitter compound. In other words, one of the ions has both the lowest oxidation potential and also the lowest negative reduction potential. In this way, the desired energy transfer state is facilitated the most efficiently. The energy levels of HOMO and LUMO can be determined experimentally by cyclic voltammetry measurements, or to an approximation theoretically by quantum-mechanical model calculations.

In a further embodiment of the present invention, the salt or system according to the invention is in the form [E₃]^{k}[E₁]^{p}[E₂]^{k}, where [E₁]^{p} is an emitter compound having the charge p and [E₂]^{k} and [E₃]^{k} are emitter compounds having the charge k, and p+2k=0. The index k is preferably equal to ±1 or ±2, more preferably ±1.

It is preferred here for at least one of the two [E₂]^{k} and [E₃]^{k} to be a fluorescent emitter compound. The other ion can be a fluorescent or phosphorescent emitter compound, but is preferably a fluorescent emitter compound. The [E₁]^{p} can likewise be a fluorescent or phosphorescent emitter compound.

Furthermore, it is preferred in accordance with the invention for one of the emitter compounds to form a macromolecule (polymer, oligomer, dendrimer), such as, for example, a dendrimer or a star-shaped molecule.

Small molecules in the sense of the present invention are not polymers, oligomers, dendrimers or mixtures thereof (blends). In particular, multiple recurring units, as typically occur in polymers, cannot be found in small molecules. The molecular weight of small molecules is typically below the molecular weight of polymers and in the region of oligomers having a small number of recurring units and below.

The molecular weight of small molecules is preferably less than or equal to 4000 g/mol, very preferably less than or equal to 3000 g/mol and very particularly preferably less than or equal to 2000/mol.

Polymers or macromolecules in the sense of the present invention have 10 to 10000, preferably 20 to 5000 and very preferably 50 to 2000 recurring units. Oligomers in the sense of the present invention preferably have 2 to 9 recurring units.

The degree of branching of the polymers and oligomers is between 0 (linear polymer with no branching) and 1 (fully branched dendrimer). The term dendrimer used herein corresponds to the definition by M. Fischer et al. in Angew. Chem., Int. Ed. 1999, 38, 885).

The molecular weight (MW) of polymers according to the invention is preferably in the range from 10000 to 2000000 g/mol, very preferably between 100000 and 1500000 g/mol and very particularly preferably between 200000 and 1000000 g/mol. The determination of MW can be carried out by means of methods which are very well known to the person skilled in the art, namely by means of GPC (gel permeation chromatography) using polystyrene as internal standard.

A blend is a mixture comprising at least one polymeric, dendrimeric or oligomeric component.

Examples of salts or systems according to the invention are given in the following overview (formulae (189) to (195))

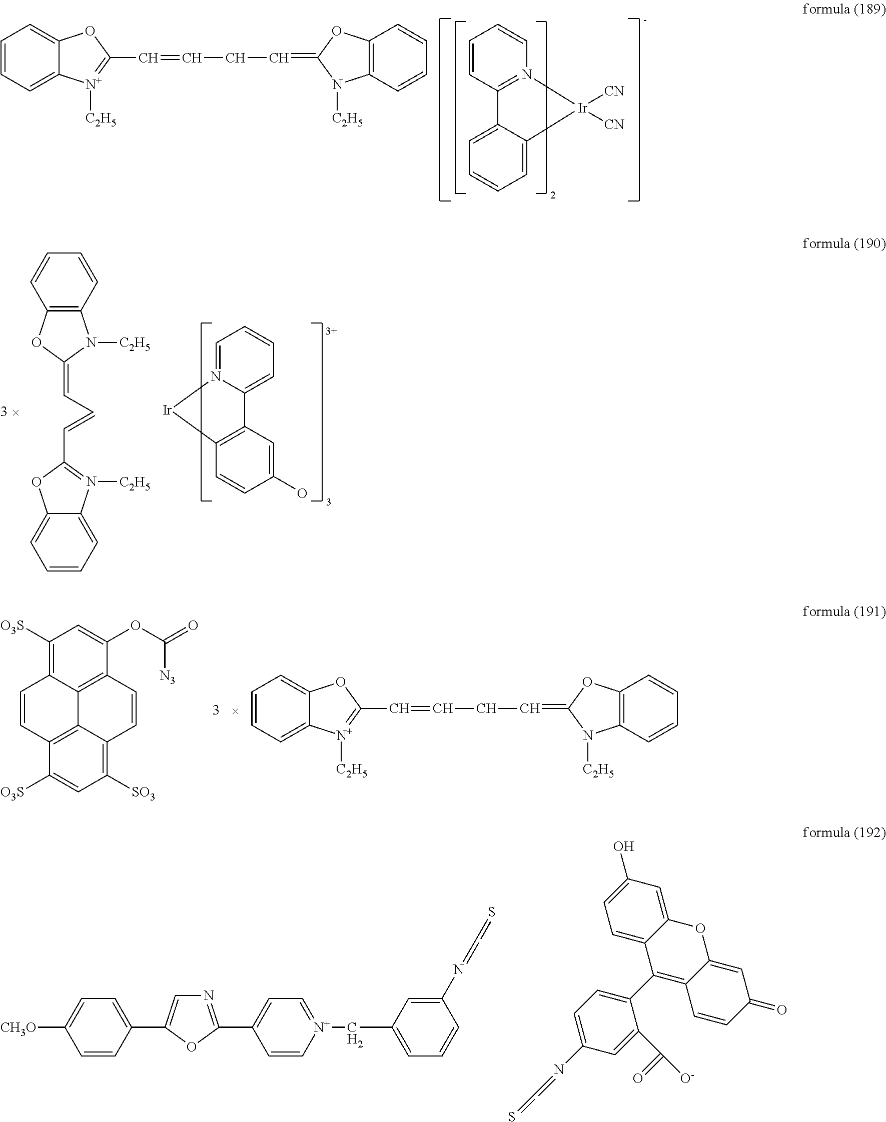

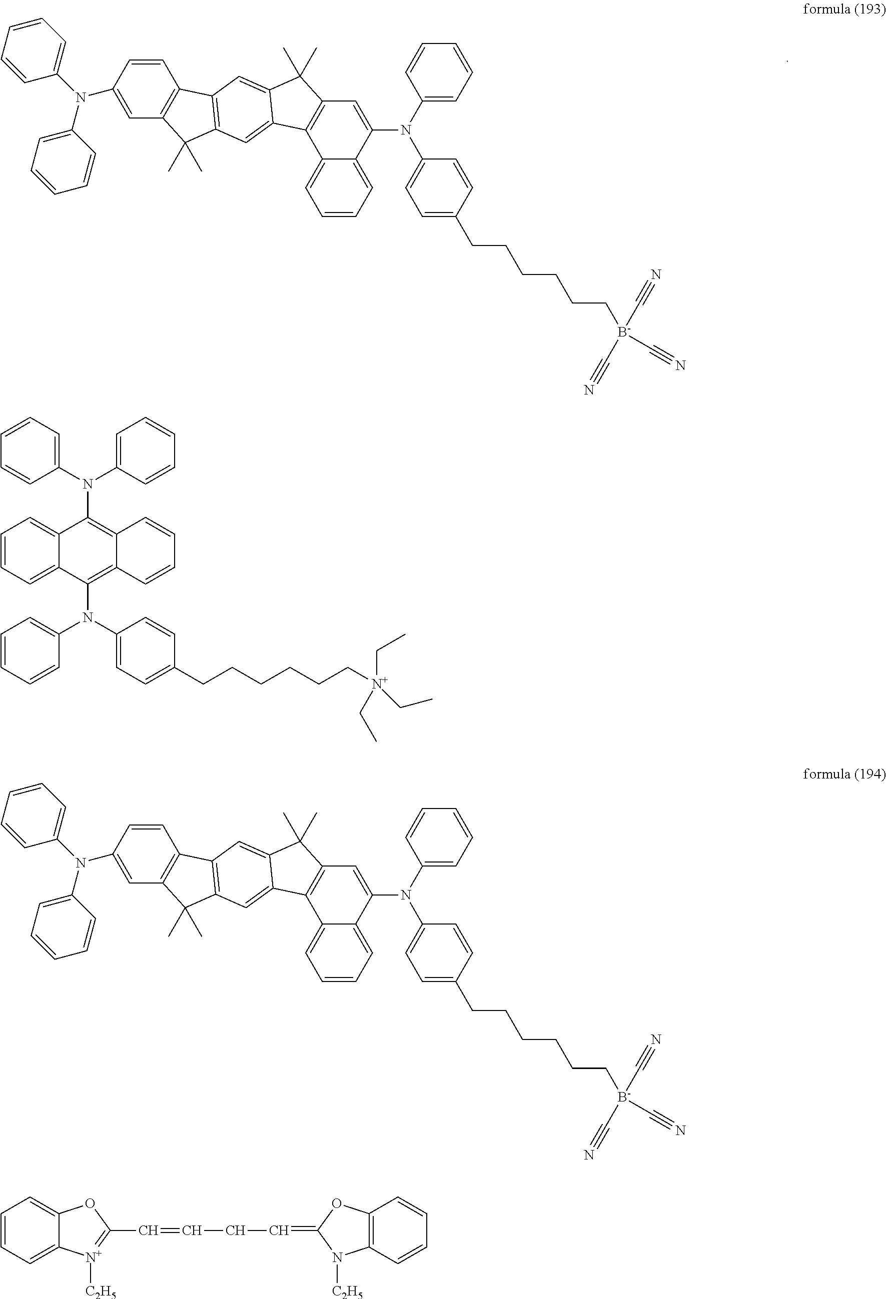 [SMILES: CC1(C)c2cc(N(c3ccccc3)c3ccccc3)ccc2-c2cc3c(cc21)-c1c(cc(N(c2ccccc2)c2ccc(CCCCCC[B-](C#N)(C#N)C#N)cc2)c2ccccc12)C3(C)C ; CC[N+](CC)(CC)CCCCCCc1ccc(N(c2ccccc2)c2c3ccccc3c(N(c3ccccc3)c3ccccc3)c3ccccc23)cc1 ; CC1(C)c2cc(N(c3ccccc3)c3ccccc3)ccc2-c2cc3c(cc21)-c1c(cc(N(c2ccccc2)c2ccc(CCCCCC[B-](C#N)(C#N)C#N)cc2)c2ccccc12)C3(C)C ; CCN1C(=CCC=Cc2oc3ccccc3[n+]2CC)Oc2ccccc21]

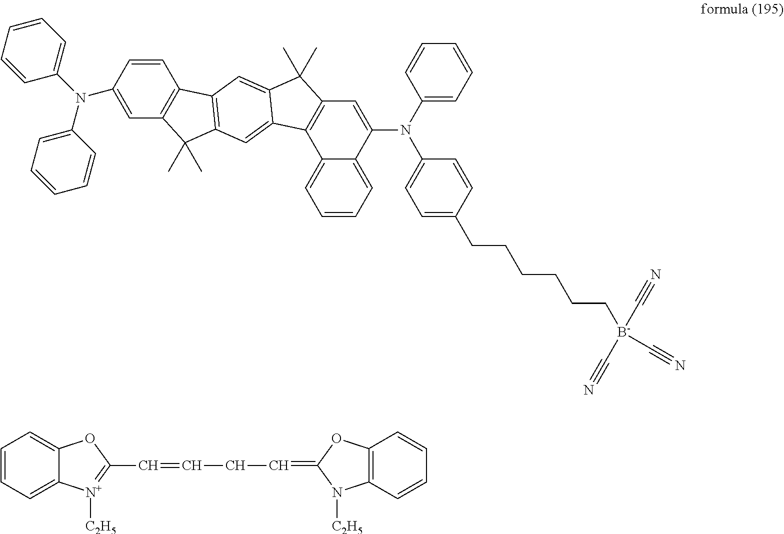 [SMILES: CC1(C)c2cc(N(c3ccccc3)c3ccccc3)ccc2-c2cc3c(cc21)-c1c(cc(N(c2ccccc2)c2ccc(CCCCCC[B-](C#N)(C#N)C#N)cc2)c2ccccc12)C3(C)C ; CCN1C(=CCC=Cc2oc3ccccc3[n+]2CC)Oc2ccccc21]

Further examples of salts according to the invention can be formed from combinations of compounds of the formulae (1) to (48), and compounds of the formulae (89) to (98) or of compounds of the formulae (1) to (48), compounds of the formulae (89) to (98) and compounds of the formulae (189) to (195).

The emission band of an emitter compound in the salt or system according to the invention is preferably in a wavelength range which overlaps with the wavelength range of the absorption band of a further emitter compound.

The maximum of the emission band of [E₁]^{p} is preferably in the wavelength region of blue light, and the maxima of the emission bands of [E₂]^{k} and [E₃]^{k} are preferably in the wavelength region of green or red light.

The emission band of [E₁]^{p} is preferably in a wavelength range which partially overlaps with the wavelength ranges of the absorption bands of [E₂]^{k} and [E₃]^{k}.

The present invention also relates to a process for the preparation of a salt or system according to the invention comprising the following steps:
(a) provision of a multiplicity of salts, where each salt contains only one emitter compound as cation or anion and where at least one emitter compound has a positive charge and at least one emitter compound has a negative charge;
(b) respective dissolution of the salts provided in a polar solvent, giving a multiplicity of solutions corresponding to the number of salts;
(c) mixing of the solutions;
(d) crystallisation of a salt which contains the at least one emitter compound having the positive charge as a cation and the at least one emitter compound having the negative charge as an anion.

The above-mentioned step (a) of the process according to the invention is to be taken to mean that each salt provided has only either one emitter compound as cation or as anion and the respective counterion thereto does not represent an emitter compound. Suitable cationic counterions here are alkali-metal or alkaline-earth metal ions. Preferred anionic counterions here are halides.

The term “respective dissolution” in step (b) is taken to mean that precisely as many solutions are prepared as salts are provided in step (a). Polar solvents, such as water, alcohols, such as methanol, ethanol, isopropanol, etc., ketones, such as acetone, 2-butanone, acetylacetone, etc., esthers, such as ethyl acetate, benzoic acid esters, etc., cyclic ethers, such as THF and dioxane, amides, such as DMF, DMAC, NMP, sulfoxides, such as DMSO, sulfones, such as slufolane, etc., are preferably used here. Water is particularly preferred as solvent. The solutions preferably have a molarity in the range from 0.001 mol/l to 10 mol/l, preferably 0.01 mol/l to 1 mol/l, particularly preferably 0.05 mol/l to 0.5 mol/l, based on the emitter salt employed in each case.

After the respective dissolution of the salts, the solutions are mixed with one another by pouring them into one another. It is generally unimportant here which solution is added to which. Preference is given to the use of salts in which the respective counterions forms a salt which is readily soluble in the corresponding solvent with one another. A salt which contains emitter compounds as cations and anions preferably crystallises out, while the counterions of the salts employed remain in solution. For further purification of the salts, one or more further recrystallisations can be carried out.

In a particularly preferred embodiment of the process according to the invention, two salts [E₁]⁺Y⁻ and [E₂]⁻X⁺ are provided in step (a), where [E₁]⁺ and [E₂]⁻ represent emitter compounds and Y⁻ and X⁺ represent corresponding counterions. In this way, a salt [E₁]⁺[E₂]⁻ is obtained in step (d). The counterions Y⁻ and X⁺ together preferably form a salt X⁺Y⁻ which is more soluble than [E₁]⁺[E₂]⁻ and thus preferably remains in solution in the corresponding solvent while [E₁]⁺[E₂]⁻ precipitates out or crystallises out.

In a further preferred embodiment of the process according to the invention, three salts [E₁]²⁺[2Y]⁻, [E₂]⁻[X]⁺ and [E₃]⁻[X]⁺ are provided in step (a), where [E₃]⁻, [E₁]²⁺ and [E₂]⁻ represent emitter compounds and Y⁻ and X⁺ represent corresponding counterions, but where X⁺ may be identical to or different from one another on each occurrence, and the case where one doubly negative counterion may also be present instead of two counterions Y⁻ is also covered in accordance with the invention. In the corresponding manner, a salt [E₃]⁻[E₁]²⁺[E₂]⁻ is obtained in step (d). The counterions Y⁻ and X⁺ together again form a salt X⁺Y⁻ which is more soluble than [E₃]⁻[E₁]²⁺[E₂]⁻.

The anions Y⁻ here can be the following: F—, Cl—, Br—, I—, etc., composite anions, such as OH—, CN—, SCN—, N3-, BF₄⁻, PF₆⁻, PO₄³⁻, SO₄³⁻, etc., organic anions, such as carboxylates, alcoholoates, thiolates, sulfonates, etc.

The cations X⁺ here can be the following: H⁺, Li⁺, Na⁺, K⁺, Cs⁺, Mg²⁺, Ca²⁺ etc., composite cations, such as NH₄⁺, PH₄⁺, etc., organic cations, such as ammonium, phosphonium, etc.

A “C₁₋₄₀-alkyl” in the present invention is preferably taken to mean linear, branched or cyclic alkyl groups. The linear alkyl groups preferably have 1 to 6, 1 to 10 or 1 to 40 carbon atoms. The branched or cyclic alkyl groups preferably have 3 to 6, 3 to 10 or 3 to 40 carbon atoms. Preference is given to alkyl groups having 1 to 6, or 3 to 6 carbon atoms, particularly preferably 1 to 3, or 3 carbon atoms. One or more hydrogen atoms on these alkyl groups may be replaced by a fluorine atom. In addition, one or more of the CH₂ groups in these units may be replaced by NR, O or S (R here is a radical selected from the group consisting of H and C₁₋₆-alkyl). If one or more of the CH₂ groups is replaced by NR, O or S, it is particularly preferred for only one of these groups to be replaced; particularly preferably by an O atom. Examples of such compounds include the following: methyl, ethyl, n-propyl, i-propyl, n-butyl, i-butyl, s-butyl, t-butyl, 2-methylbutyl, n-pentyl, s-pentyl, n-hexyl, n-heptyl, n-octyl, 2-ethylhexyl, trifluoromethyl, pentafluoroethyl, 2,2,2-trifluoroethyl, cyclopentyl, cyclohexyl, cycloheptyl and cyclooctyl. C₁₋₄-alkyl, C₁₋₁₀-alkyl and C₁₋₂₀-alkyl are likewise taken to mean alkyl groups as defined above, with the proviso that they contain correspondngly fewer carbon atoms.

A mono- or polycyclic aromatic or heteroaromatic hydrocarbon radical preferably contains 5 to 40, more preferably 5 to 20, most preferably 5 or 6 aromatic ring atoms. If the unit is an aromatic unit, it preferably contains 6 to 40, more preferably 6 to 20, most preferably 6 carbon atoms as ring atoms. If the unit is a heteroaromatic unit, it contains 5 to 40, more preferably 5 to 10, most preferably 5 aromatic ring atoms, at least one of which is a heteroatom. The heteroatoms are preferably selected from N, O and/or S. An aromatic or heteroaromatic unit here is taken to mean either a simple aromatic ring, i.e. benzene, or a simple heteroaromatic ring, for example pyridine, pyrimidine, thiophene, etc., or a condensed aryl or heteroaryl group, for example naphthalene, anthracene, phenanthrene, quinoline, isoquinoline, benzothiophene, benzofuran and indole, etc.

Examples according to the invention of the aromatic or heteroaromatic hydrocarbon radicals are accordingly: benzene, naphthalene, anthracene, phenanthrene, pyrene, chrysene, benzanthracene, perylene, naphthacene, pentacene, benzopyrene, furan, benzofuran, isobenzofuran, dibenzofuran, thiophene, benzothiophene, isobenzothiophene, dibenzothiophene, pyrrole, indole, isoindole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

A mono- or polycyclic aromatic ring system in the sense of this invention is preferably taken to mean an aromatic ring system having 6 to 60 carbon atoms, preferably 6 to 30, particularly preferably 6 to 10 carbon atoms. An aromatic ring system in the sense of the present invention is intended to be taken to mean a system which does not necessarily contain only aromatic groups, but instead in which, in addition, a plurality of aromatic may be interrupted by a short non-aromatic unit (less than 10% of the atoms other than H, preferably less than 5% of the atoms other than H), such as, for example, sp³-hybridised C, O, N, etc. These aromatic ring systems may be monocyclic or polycyclic, i.e. they may contain one ring (for example phenyl) or two or more rings, which may also be condensed (for example naphthyl) or covalently linked (for example biphenyl), or contain a combination of condensed and linked rings.

Preferred aromatic ring systems are, for example, phenyl, biphenyl, triphenyl, naphthyl, anthracyl, binaphthyl, phenanthryl, dihydrophenanthryl, pyrene, dihydropyrene, chrysene, perylene, tetracene, pentacene, benzopyrene, fluorene and indene.

A mono- or polycyclic heteroaromatic ring system in the sense of this invention is preferably taken to mean a heteroaromatic ring system having 5 to 60 ring atoms, preferably 5 to 30, particularly preferably 5 to 14 ring atoms. The heteroaromatic ring system contains at least one heteroatom selected from N, O and S (remaining atoms are carbon). A heteroaromatic ring system is additionally intended to be taken to mean a system which does not necessarily contain only aromatic or heteroaromatic groups, but instead in which, in addition, a plurality of aromatic or heteroaromatic groups may be interrupted by a short non-aromatic unit (<10% of the atoms other than H, preferably <5% of the atoms other than H), such as, for example, sp³-hybridised C, O, N, etc. These heteroaromatic ring systems may be monocyclic or polycyclic, i.e. they may contain one ring (for example pyridyl) or two or more rings, which may also be condensed or covalently linked, or contain a combination of condensed and linked rings.

Preferred heteroaromatic ring systems are, for example, 5-membered rings, such as pyrrole, pyrazole, imidazole, 1,2,3-triazole, 1,2,4-triazole, tetrazole, furan, thiophene, selenophene, oxazole, isoxazole, 1,2-thiazole, 1,3-thiazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 6-membered rings, such as pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, or condensed groups, such as indole, isoindole, indolizine, indazole, benzimidazole, benzotriazole, purine, naphthimidazole, phenanthrimidazole, pyridimidazole, pyrazinimidazole, quinoxalinimidazole, benzoxazole, naphthoxazole, anthroxazole, phenanthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]thiophene, dithienothiophene, isobenzothiophene, dibenzothiophene, benzothiadiazothiophene or combinations of these groups. Particular preference is given to imidazole, benzimidazole and pyridine.

The term “aryl” or “aryl group” is taken to mean a mono- or polycyclic aromatic or heteroaromatic ring system, as defined above.

The term “alkylsilyl” is taken to mean mono(C₁₋₁₂-alkyl)silyl groups, di(C₁₋₁₂-alkyl)silyl groups and tri-(C₁₋₁₂-alkyl)silyl groups.

A “mono(C₁₋₁₂-alkyl)silyl group” in the present invention is taken to mean an (SiH₂) group which is linked to a linear or branched alkyl group (as defined above) having 1 or 3 to 12 carbon atoms respectively, more preferably 1 or 3 to 6 carbon atoms respectively. A “di(C₁₋₁₂-alkyl)silyl group” in the present invention is taken to mean an (SiH) unit which is linked to two linear or branched alkyl groups (as defined above), on each occurrence identical or different, having 1 or 3 to 12 carbon atoms respectively, particularly preferably 1 or 3 to 6 carbon atoms respectively. A “tri(C₁₋₁₂-alkyl)-silyl group” in the present invention is taken to mean an (Si) unit which is linked to three linear or branched alkyl groups (as defined above), on each occurrence identical or different, having 1 or 3 to 12 carbon atoms respectively, more preferably 1 or 3 to 6 carbon atoms respectively. The examples indicated above in connection with “C₁₋₄₀-alkyl group” also apply to the alkyl groups present here, so long as they have the corresponding number of carbon atoms.

“Silyl” in the present compound is taken to mean a silyl group having 1 or 3 to 5 silicon atoms, which is linear or branched. Examples thereof are monosilyl, disilyl, trisilyl, tetrasilyl and pentasilyl.

“Arylsilyl” in the present invention is taken to mean an Si₁-silyl group which is substituted by one, two or three, mono- or polycyclic, aromatic or heteroaromatic ring systems having 5 to 60 aromatic ring atoms.

“Alkoxyalkyl” in the present invention is taken to mean a monovalent ether unit having two linear or branched alkyl groups having 1 or 3 to 12, more preferably 1 or 3 to 6 carbon atoms respectively, which are bonded via an oxygen atom. The examples indicated above in connection with the definition of “C₁₋₄₀-alkyl” also apply here to the alkyl groups present, so long as they have the corresponding number of atoms.

“Arylalkoxyalkyl” in the present invention is taken to mean a monovalent unit as defined above for “alkoxyalkyl”, where one alkyl group is substituted by an aryl which represents a mono- or polycyclic, aromatic or hetero-aromatic ring system having 5 to 60 aromatic ring atoms as defined above.

“Alkylthioalkyl” in the present invention is taken to mean a monovalent thioether unit having two linear or branched alkyl groups having 1 or 3 to 12, more preferably 1 or 3 to 6 carbon atoms respectively, which are bonded via a sulfur atom. The examples indicated above in connection with the definition of “C₁₋₄₀-alkyl” also apply here to the alkyl groups present, so long as they have the corresponding number of atoms.

“Alkyl sulfone” in the present invention is taken to mean an S(═O)₂— unit which is substituted by an alkyl group having 1 to 12 carbon atoms. The examples indicated above in connection with the definition of “C₁₋₄₀-alkyl” also apply here to the alkyl groups present so long as they have the corresponding number of atoms.

“C₁₋₁₂-alkyl sulfoxide” in the present invention is taken to mean an —S(═O)— unit which is substituted by an alkyl group having 1 to 12 carbon atoms. The examples indicated above in connection with the definition of “C₁₋₄₀-alkyl” also apply here to the alkyl groups present so long as they have the corresponding number of atoms.

The present invention furthermore relates to a multilayer structure which comprises a layer which comprises a salt or system according to the invention.

A multilayer structure in the present invention is taken to mean a multilayer structure comprising two or more layers, which are preferably applied successively to a glass support. The layers may comprise individual compounds according to the invention. It is preferred for the layers to comprise further compounds, polymers or oligmers having different properties.

The present invention furthermore relates to a formulation, in particular a solution, dispersion or emulsion, comprising at least one salt or system according to the invention and at least one solvent. Solvents which can be employed are all conceivable ones which are capable of dissolving the salts or systems according to the invention or forming a suspension with them. The following solvents are preferred here in accordance with the invention: dichloromethane, trichloromethane, monochlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 1,1,1-trichloroethane, 1,1,2,2-tetrachloroethane, ethyl acetate, n-butyl acetate, dimethylformamide, dimethylacetamide, dimethyl sulfoxide, tetralin, decalin, indane and/or mixtures thereof.

The concentration of the salt according to the invention in the solution is preferably 0.1 to 10% by weight, more preferably 0.5 to 5% by weight, based on the total weight of the solution. The solution optionally also comprises one or more binders in order to adjust the rheological properties of the solution correspondingly, as described, for example, in WO 2005/055248 A1.

After appropriate mixing and ageing of the solutions, these are divided into one of the following categories: “full” solution, “borderline” solution or insoluble. The border line is drawn between these categories with reference to the solubility parameters. The corresponding values can be obtained from the literature, such as, for example, from “Crowley, J. D., Teague, G. S. Jr. and Lowe, J. W. Jr., Journal of Paint Technology, 38, No. 496, 296 (1966)”.

Solvent mixtures can also be used and are identified as described in “Solvents, W. H. Ellis, Federation of Societies for Coatings Technology, pp. 9 to 10, 1986”. Processes of this type can result in a mixture of so-called “non”-solvents which dissolve the composition, although it is desirable to have at least one true solvent in the mixture.

A further preferred form of the formulation is an emulsion, and more preferably a miniemulsion, which are prepared, in particular, as heterophase systems, in which stable nanodroplets of a first phase are dispersed in a second continuous phase.

Both a miniemulsion, in which the continuous phase is a polar phase, and also an inverse miniemulsion, in which the continuous phase is a non-polar phase, can be used in the present invention. The preferred form is a miniemulsion. In order to increase the kinetic stability of the emulsion, surfactants can also be admixed. The choice of the solvents for two-phase systems, the surfactants and the processing to give a stable miniemulsion should be known to a person skilled in the art in this area on the basis of his expert knowledge or through numerous publications, such as, for example, a comprehensive article by Landfester in Annu. Rev, Mater. Res. (06), 36, p. 231.

For use of so-called thin layers in electronic or opto-electronic devices, the salt according to the invention or a formulation thereof can be deposited by a correspondingly suitable process. Liquid coating of devices, such as, for example, of OLEDs, is more desirable than vacuum deposition techniques. Deposition methods from solution are particularly preferred. Preferred deposition techniques include, without correspondingly restricting the invention, dip coating, spin coating, ink-jet printing, letterpress printing, screen printing, doctor blade coating, roller printing, reverse roller printing, offset lithography, flexographic printing, web printing, spray coating, brush coating or pad printing and slot-die coating. Ink-jet printing is particularly preferred and enables the production of high-resolution displays.

The solutions according to the invention can be applied to prefabricated device substrates with the aid of ink-jet printing or by microdispensing. To this end, preference is given to the use of industrial piezoelectric print heads, such as from Aprion, Hitachie-Koki, Inkjet Technology, On Target Technology, Picojet, Spectra, Trident, Xaar, in order to apply the organic semiconductor layers to a substrate. In addition, semi-industrial print heads, such as those from Brother, Epson, Konika, Seiko Instruments, Toshiba TEC or single-nozzle microdispensing equipment, as manufactured, for example, by Mikrodrop and Mikrofab, can also be used.

In order that the salt according to the invention can be applied by ink-jet printing or microdispensing, it should first be dissolved in a suitable solvent. The solvents must meet the above-mentioned requirements and must not have any disadvantageous effects on the print head selected. In addition, the solvents should have a boiling point of above 100° C., preferably above 140° C. and more preferably above 150° C., in order to avoid processing problems caused by drying-out of the solution inside the print head. Besides the above-mentioned solvents, the following solvents are also suitable: substituted and unsubstituted xylene derivatives, di-C₁₋₂-alkyl-formamides, substituted and unsubstituted anisoles and other phenol ether derivatives, substituted heterocycles, such as substituted pyridines, pyrapsines, pyrimidines, pyrrolidinones, substituted and unsubstituted N,N-di-C₁₋₂-alkylanilines and other fluorinated or chlorinated aromatic compounds.

A preferred solvent for the deposition of the salt according to the invention by ink-jet printing comprises a benzene derivative which contains a benzene ring which is substituted by one or more substituents, in which the total number of carbon atoms of the one or more substituents is at least three. Thus, for example, the benzene derivative may be substituted by a propyl group or three methyl groups, where in each case the total number of carbon atoms must be at least three. A solvent of this type enables the formation of an ink-jet liquid which comprises the solvent with the salt according to the invention, and reduces or prevents clogging of the nozzles and separation of the components during spraying. The solvent(s) can be selected from the following example list: dodecylbenzene, 1-methyl-4-tert-butylbenzene, terpineollimonene, isodurol, terpinolene, cymol and dethylbenzene. The solvent may also be a solvent mixture comprising two or more solvents, where each of the solvents preferably has a boiling point of greater than 100° C., more preferably greater than 140° C. Solvents of this type promote film formation of the deposited layer and reduce layer errors.

The ink-jet liquid, (i.e. a mixture, preferably of solvent(s), binder and the compound according to the invention) preferably has a viscosity at 20° C. of 1 to 100 mPa·s, more preferably 1 to 50 mPa·s and most preferably 1 to 30 mPa·s.

The salt or formulation according to the invention may additionally comprise one or more further components, such as, for example, surface-active substances, lubricants, wetting agents, dispersants, water-repellent agents, adhesives, flow improvers, antifoaming agents, air deposition agents, diluents, which may be reactive or unreactive substances, assistants, colorants, dyes or pigments, sensitisers, stabilisers or inhibitors.

A formulation according to the invention cannot only be prepared by dissolving a salt according to the invention obtained as a solid in a solvent, but also by carrying out process steps (a) to (c) mentioned above, and subsequently removing the undesired counterions from the solution by chromatographic methods or ion-exchange processes.

The invention furthermore relates to the use of the above-mentioned salts or systems according to the invention in an organic electroluminescent device. The salts or systems according to the invention are preferably formed here as or in an electroluminescent layer. A layer is preferably formed by applying a formulation according to the invention to a support and subsequently removing the solvent.

In a further preferred embodiment, the formulation comprises at least one further neutral organic functional compound selected from HTM (hole transport material), ETM (electron transport material), host and emitters.

The present invention furthermore relates to an electronic device containing a salt or system according to the invention or formulation.

The electronic device is preferably an organic electroluminescent device, preferably comprising a cathode, an anode and at least one organic layer, where the organic layer comprises the salt or system according to the invention or formulation.

As just stated, the organic layer which comprises the salt or system or formulation according to the invention is preferably the emitting layer. In a very preferred embodiment, the organic layer comprises at least one further neutral organic functional compound selected from HTM (hole transport material), ETM (electron tranpsort material), host and emitters, particularly preferably at least one host material.

Suitable host materials for this purpose are materials from various classes of substance. Preferred host materials are selected from the classes of the oligoarylenes (for example 2,2′,7,7-tetraphenylspirobifluorene in accordance with EP 676461 or dinaphthylanthracene), in particular the oligoarylenes containing condensed aromatic groups, such as, for example, anthracene, benzanthracene, benzophenanthrene (DE 102009005746.3, WO 2009/069566), phenanthrene, tetracene, corones, chrysene, fluorene, spirofluorene, perylene, phthaloperylene, naphthaloperylene, decacyclene, rubrene, the oligoarylenevinylenes (for example DPVBi=4,4′-bis(2,2-diphenylethenyl)-1,1′-biphenyl) or spiro-DPVBi in accordance with EP 676461), the polypodal metal complexes (for example in accordance with WO 04/081017), in particular metal complexes of 8-hydroxyquinoline, for example AlQ₃ (=aluminium(III) tris(8-hydroxyquinoline)) or bis(2-methyl-8-quinolinolato)-4-(phenylphenolinolato)aluminium, also with imidazole chelate (US 2007/0092753 A1) and the quinoline-metal complexes, aminoquinoline-metal complexes, benzoquinoline-metal complexes, the hole-transporting compounds (for example in accordance with WO 2004/058911), the electron-transporting compounds, in particular ketones, phosphine oxides, sulfoxides, etc. (for example in accordance with WO 2005/084081 and WO 2005/084082), the atropisomers (for example in accordance with WO 2006/048268), the boronic acid derivatives (for example in accordance with WO 2006/117052) or the benzanthracenes (for example in accordance with WO 2008/145239).

Particularly preferred host materials are selected from the classes of the oligoarylenes, comprising anthracene, benzanthracene and/or pyrene or atropisomers of these compounds. An oligoarylene in the sense of this invention is intended to be taken to mean a compound in which at least three aryl or arylene groups are bonded to one another.

Preferred host materials are selected, in particular, from compounds of the formula (195a),

Ar⁴—(Ar⁵)ₚ—Ar⁶ formula (195a)

where Ar⁴, Ar⁵, Ar⁶ is on each occurrence, identically or differently, an aryl or heteroaryl group having 5 to 30 aromatic ring atoms, which may be substituted by one or more radicals R¹, and R1 and p have the same meaning as described above; the sum of the π electrons in Ar⁴, Ar⁵ and Ar⁶ is at least 30 if p=1, and at least 36 if p=2, and at least 42 if p=3.

In the host materials of the formula (195a), the group Ar⁵ particularly preferably stands for anthracene, which may be substituted by one or more radicals R¹, and the groups Ar⁴ and Ar⁶ are bonded in the 9- and 10-position. At least one of the groups Ar⁴ and/or Ar⁶ is very particularly preferably a condensed aryl group selected from 1- or 2-naphthyl, 2-, 3- or 9-phenanthrenyl or 2-, 3-, 4-, 5-, 6- or 7-benzanthracenyl, each of which may be substituted by one or more radicals R¹. Anthracene-based compounds are described in US 2007/0092753 A1 and US 2007-0252517 A1, for example 2-(4-methylphenyl)-9,10-di(2-naphthyl)anthracene, 9-(2-naphthyl)-10-(1,1′-biphenyl)anthracene and 9,10-bis[4-(2,2-diphenylethenyl)phenyl]anthracene, 9,10-diphenylanthracene, 9,10-bis(phenylethynyl)anthracene and 1,4-bis(9′-ethynylanthracenyl)benzene. Preference is also given to compounds having two anthracene units (US 2008/0193796 A1), for example 10, 10′-bis[1,1′,4′, 1″]terphenyl-2-yl-9,9′-bisanthracenyl.

Further preferred compounds are derivatives of arylamine, styrylamine, fluorescein, diphenylbutadiene, tetraphenylbutadiene, cyclopentadienes, tetraphenylcyclopentadiene, pentaphenylcyclopentadiene, coumarin, oxadiazole, bisbenzoxazoline, oxazole, pyridine, pyrazine, imine, benzothiazole, benzoxazole, benzimidazole (US 2007/0092753 A1), for example 2,2′,2″-(1,3,5-phenylene)tris[1-phenyl-1H-benzimidazole], aldazine, stilbene, styrylarylene derivatives, for example 9,10-bis[4-(2,2-diphenylethenyl)phenyl]anthracene and distyrylarylene derivatives (U.S. Pat. No. 5,121,029), diphenylethylene, vinylanthracene, diaminocarbazole, pyran, thiopyran, diketopyrrolopyrrole, polymethine, cinnamic acid esters and fluorescent dyes.

Particular preference is given to derivatives of arylamine and styrylamine, for example TNB (=4,4′-bis[N-(1-naphthyl)-N-(2-naphthyl)amino]biphenyl). Metal-oxinoid complexes, such as LiQ or AIQ₃, can be used as co-hosts.

Preferred compound with oligoarylene as matrix:

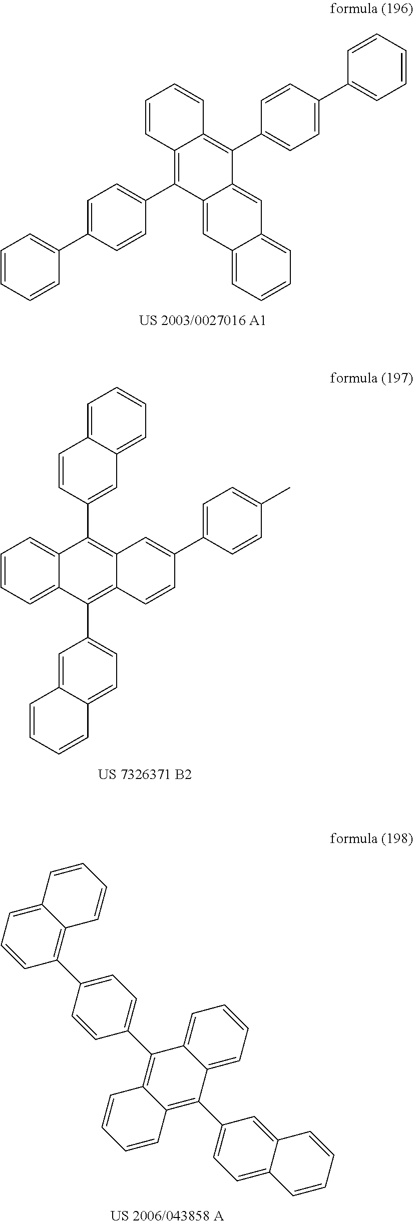

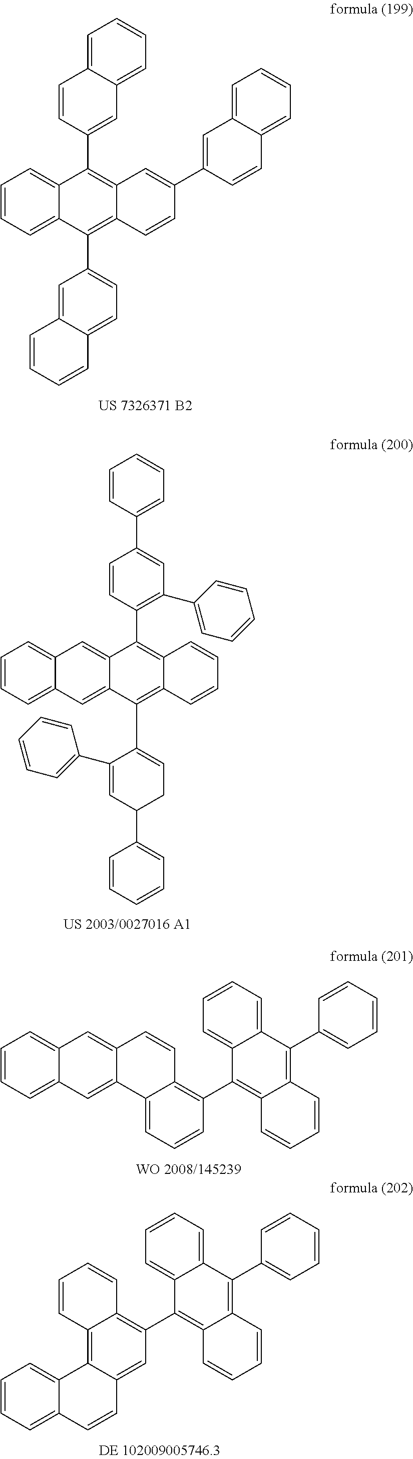

Suitable matrix materials in electronic devices for the salt according to the invention are CBP (N,N-biscarbazolylbiphenyl), carbazole derivatives (for example in accordance with WO 2005/039246, US 2005/0069729, JP 2004/288381, EP 1205527 or WO 2008/086851), azacarbazoles (for example in accordance with EP 1617710, EP 1617711, EP 1731584, JP 2005/347160), ketones (for example in accordance with WO 2004/093207 or in accordance with DE 102008033943.1), phosphine oxides, sulfoxides and sulfones (for example in accordance with WO 2005/003253), oligophenylenes, aromatic amines (for example in accordance with US 2005/0069729), bipolar matrix materials (for example in accordance with WO 2007/137725), silanes (for example in accordance with WO 2005/111172), 9,9-diarylfluorene derivatives (for example in accordance with DE 102008017591), azaboroles or boronic esters (for example in accordance with WO 2006/117052), triazine derivatives (for example in accordance with DE 102008036982), indolocarbazole derivatives (for example in accordance with WO 2007/063754 or WO 2008/056746), indenocarbazole derivatives (for example in accordance with the unpublished application DE 102009023155.2 and DE 102009031021.5), diazaphosphole derivatives (for example in accordance with the unpublished application DE 102009022858.6), triazole derivatives, oxazoles and oxazole derivatives, imidazole derivatives, polyarylalkane derivatives, pyrazoline derivatives, pyrazolone derivatives, distyrylpyrazine derivatives, thiopyran dioxide derivatives, phenylenediamine derivatives, tertiary aromatic amines, styrylamines, amino-substituted chalcone derivatives, indoles, hydrazone derivatives, stilbene derivatives, silazane derivatives, aromatic dimethylidene compounds, carbodiimide derivatives, metal complexes of 8-hydroxyquinoline derivatives, such as, for example, AlQ₃, the 8-hydroxyquinoline complexes may also contain triarylaminophenol ligands (US 2007/0134514 A1), metal complex-polysilane compounds, and thiophene, benzothiophene and dibenzothiophene derivatives.

Examples of preferred carbazole derivatives are mCP (=1,3-N,N-dicarbazolebenzene (=9,9′-(1,3-phenylene)bis-9H-carbazole), formula (203), US 2005/0249976), CDBP (=9,9′-(2,2′-dimethyl[1,1′-biphenyl]-4,4′-diyl)bis-9H-carbazole), 1,3-bis(N,N′-dicarbazole)benzene (=1,3-bis(carbazol-9-yl)-benzene), OPVK (polyvinylcarbazole), 3,5-di(9H-carbazol-9-yl)biphenyl and the further compounds having the formulae (204) to (207) depicted below (see also US 2007/0128467, US 2007/0128467).

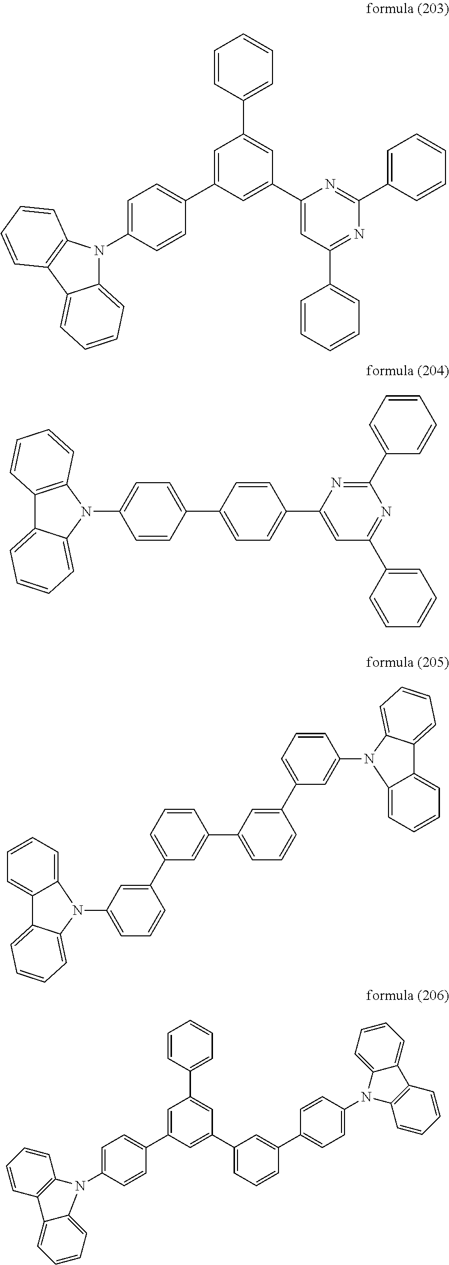 [SMILES: c1ccc(-c2cc(-c3ccc(-n4c5ccccc5c5ccccc54)cc3)cc(-c3cc(-c4ccccc4)nc(-c4ccccc4)n3)c2)cc1 ; c1ccc(-c2cc(-c3ccc(-c4ccc(-n5c6ccccc6c6ccccc65)cc4)cc3)nc(-c3ccccc3)n2)cc1 ; c1cc(-c2cccc(-c3cccc(-n4c5ccccc5c5ccccc54)c3)c2)cc(-c2cccc(-n3c4ccccc4c4ccccc43)c2)c1 ; c1ccc(-c2cc(-c3ccc(-n4c5ccccc5c5ccccc54)cc3)cc(-c3cccc(-c4ccc(-n5c6ccccc6c6ccccc65)cc4)c3)c2)cc1]

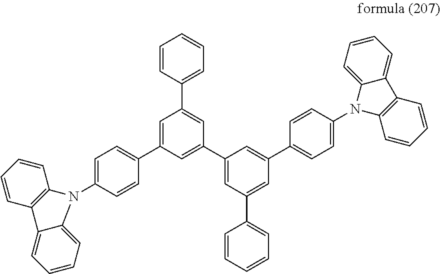 [SMILES: c1ccc(-c2cc(-c3ccc(-n4c5ccccc5c5ccccc54)cc3)cc(-c3cc(-c4ccccc4)cc(-c4ccc(-n5c6ccccc6c6ccccc65)cc4)c3)c2)cc1]

Further preferred matrix materials in the sense of the present invention are Si tetraaryl compounds as disclosed, for example, in US 004/209115, US 2004/0209116 US 2007/0087219, US 2007/0087219 and H. Gilman, E. A. Zuech, Chemistry & Industry (London, United Kingdom), 1960, 120, particular preference is given here to the compounds of the formulae (208) to (215).

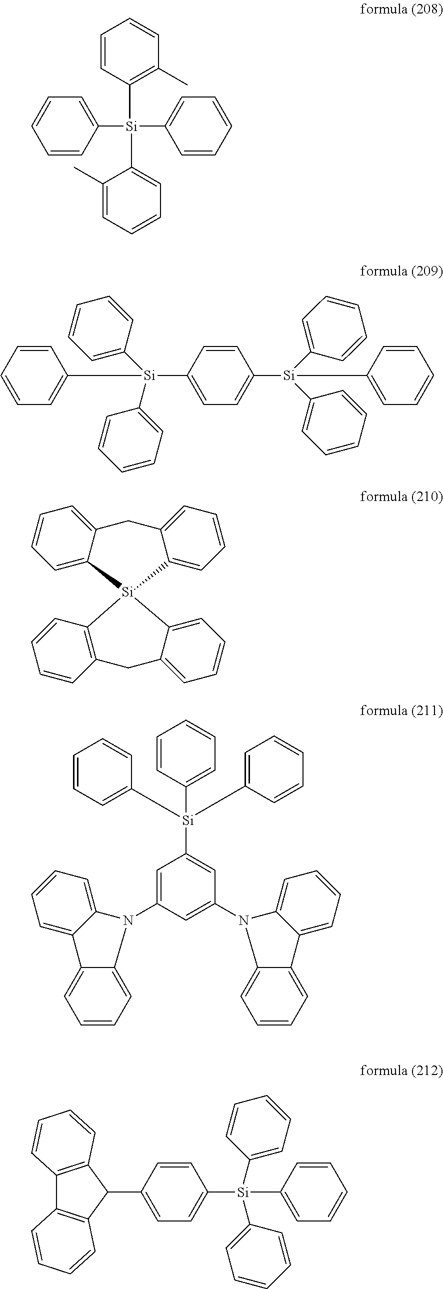

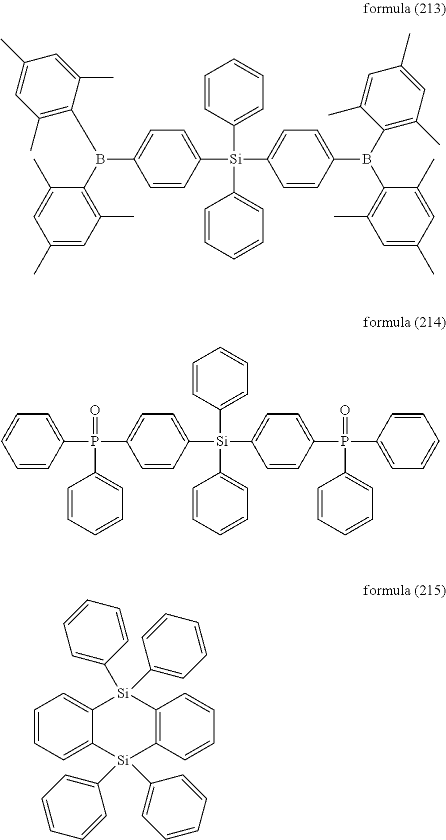

Particularly preferred matrix materials for phosphorescent dopants are compounds in EP 652273, DE 102009022858.6, DE 102009023155.2, WO 2007/063754 and WO 2008/056746, in particular the compounds of the formulae (216) to (219).

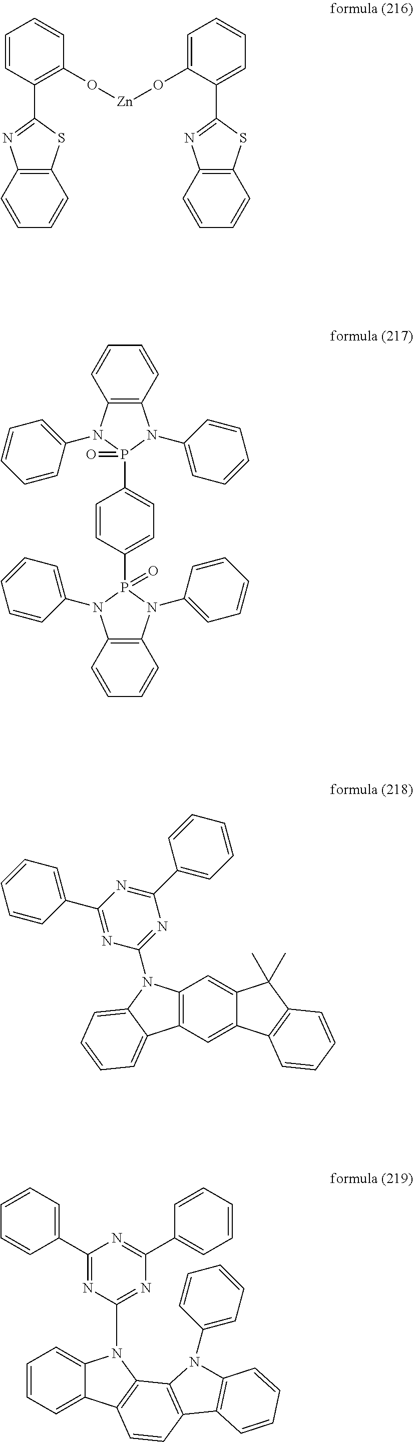

The organic electroluminescent device may in addition comprise further layers selected from in each case one or more hole-injection layers, hole-transport layers, hole-blocking layers, electron-transport layers, electron-injection layers, electron-blocking layers, charge-generation layers and/or layers which generate organic or inorganic P/N junctions. The electroluminescent device may in addition comprise further emitting layers. So-called interlayers, which have, for example, an exciton-blocking function, are preferably introduced between two emitting layers. However, it should be pointed out that each of these layers does not necessarily have to be present.

The organic electroluminescent device which comprises the salt or system according to the invention preferably has a planar shape and/or is in the form of a fibre.

A fibre in the sense of the present invention is taken to mean any shape in which the ratio between length to diameter is greater than or equal to 10:1, preferably 100:1, where the shape of the cross section along the longitudinal axis is not important. The cross section along the longitudinal axis may accordingly be, for example, round, oval, triangular, rectangular or polygonal. Light-emitting fibres have preferred properties with respect to their use. Thus, they are suitable, inter alia, for use in the area of therapeutic and/or cosmetic phototherapy. Further details in this respect are described in the prior art (for example in U.S. Pat. No. 6,538,375, US 2003/0099858, Brenndan O'Connor et al. (Adv. Mater. 2007, 19, 3897-3900 and the unpublished patent application EP 10002558.4).

If the organic electroluminescent device comprises a plurality of emitting layers, where at least one emitting layer comprises the salt or system according to the invention, these plurality of layers preferably have in total a plurality of emission maxima between 380 nm and 750 nm, resulting overall in white emission, i.e. various emitting compounds which are able to fluoresce or phosphoresce are used in the emitting layers. Particular preference is given to three layer systems, where the three layers exhibit blue, green and orange or red emission, for the basic structure see, for example, WO 2005/011013.

The various layers can be applied differently for the purposes of the invention. For example, one or more layers in the electroluminescent device according to the invention can be applied from solution and one or more layers can be applied via a sublimation process, in which the materials are applied by vapour deposition in vacuum sublimation units at a pressure <10⁻⁵ mbar, preferably <10⁻⁶ mbar, particularly preferably <10⁻⁷ mbar. It is likewise possible to apply one or more layers by means of OVPD (organic vapour phase deposition) processes or with the aid of carrier-gas sublimation, in which the materials are applied at a pressure between 10⁻⁵ mbar and 1 bar. A special case of this process is the OVJP (organic vapour jet printing) process, in which the materials are applied directly through a nozzle and are thus structured (for example M. S. Arnold et al., Appl. Phys. Lett. 2008, 92, 053301).

However, it is particularly preferred for one or more layers in the organic electroluminescent device to be applied from solution, for example by spin coating or by means of any desired printing process, such as, for example, screen printing, flexographic printing or offset printing. But particularly preferably LITI (light induced thermal imaging, thermal transfer printing), or ink-jet printing. These processes are generally known to the person skilled in the art and can be applied by him without problems to organic electroluminescent devices. For application of these processes, it is particularly preferred for the salt or system according to the invention to be soluble in a polar solvent, such as, for example, water, acetonitrile, acetone, tetra-hydrofuran, dimethylformamide, cyclohexanone or dimethyl sulfoxide, or in a usual solvent, such as, for example, toluene, chlorobenzene, anisole, etc., or a solvent mixture thereof. In this way, it is possible to prepare a miniemulsion which consists of a continuous phase and microdroplets. The continuous phase preferably comprises a nonpolar solvent in which the solution of the salt according to the invention are in the form of nano-droplets. The nonpolar solvent preferably comprises a compound which represents a matrix material for the salt according to the invention. In this way, it is possible to produce, for example after removal of the solvents, a layer in which the emitter compounds of the salt or system according to the invention is embedded as guest molecules into the compounds forming the matrix material.

The device usually comprises a cathode and an anode (electrodes). The electrodes (cathode, anode) are selected for the purposes of this invention in such a way that their potential corresponds as closely as possible to the potential of the adjacent organic layer in order to ensure the most efficient electron or hole injection possible.

The cathode preferably comprises metal complexes, metals having a low work function, metal alloys or multilayered structures comprising various metals, such as, for example, alkaline-earth metals, alkali metals, main-group metals or lanthanoids (for example Ca, Ba, Mg, Al, In, Mg, Yb, Sm, etc.). In the case of multilayered structures, further metals which have a relatively high work function, such as, for example, Ag, may also be used in addition to the said metals, in which case combinations of the metals, such as, for example, Ca/Ag or Ba/Ag, are generally used. It may also be preferred to introduce a thin interlayer of a material having a high dielectric constant between a metallic cathode and the organic semiconductor. Suitable for this purpose are, for example, alkali metal or alkaline-earth metal fluorides, but also the corresponding oxides (for example LiF, Li₂O, BaF₂, MgO, NaF, etc.). The layer thickness of this layer is preferably between 1 and 10 nm, more preferably 2-8 nm.

The anode preferably comprises materials having a high work function. The anode preferably has a potential of greater than 4.5 eV vs. vacuum. Suitable for this purpose are on the one hand metals having a high redox potential, such as, for example, Ag, Pt or Au. On the other hand, metal/metal oxide electrodes (for example Al/Ni/NiOₓ, Al/PtOₓ) may also be preferred. For some applications, at least one of the electrodes must be transparent in order to enable either irradiation of the organic material (O-SCs) or the coupling-out of light (OLEDs/PLEDs, O-lasers). A preferred structure uses a transparent anode. Preferred anode materials here are conductive mixed metal oxides. Particular preference is given to indium tin oxide (ITO) or indium zinc oxide (IZO). Preference is furthermore given to conductive doped organic materials, in particular conductive doped polymers.

The device is correspondingly structured in a manner known per se, depending on the application, provided with contacts and finally hermetically sealed, since the lifetime of devices of this type is drastically shortened in the presence of water and/or air.

The organic electroluminescent device according to the invention is preferably selected from the group consisting of organic electroluminescent devices (OLEDs), organic field-effect transistors (O-FETs), organic thin-film transistors (O-TFTs), organic light-emitting transistors (O-LETs), organic integrated circuits (O-ICs), organic solar cells (O-SCs), organic field-quench devices (O-FQDs), light-emitting electrochemical cells (OLECs), organic photoreceptors, “organic plasmon emitting devices” or organic laser diodes (O-lasers). Particular preference is given to organic electroluminescent devices.

The salts according to the invention can furthermore be used in therapeutic and cosmetic devices in order to treat diseases and/or to protect cosmetic effects against light or radiation.

The present invention therefore furthermore relates to the use of the salts according to the invention, formulations and devices containing the salts for the treatment, prophylaxis and diagnosis of diseases. The present invention still furthermore relates to the use, of the compounds according to the invention and devices containing the salts for the treatment and prophylaxis of cosmetic conditions.

The present invention furthermore relates to the salts according to the invention for the production of devices for the therapy, prophylaxis and/or diagnosis of therapeutic diseases.

Many diseases are associated with cosmetic aspects. Thus, a patient with severe acne in the facial area suffers not only from the medical causes and consequences of the disease, but also from the cosmetic accompanying circumstances.

Phototherapy or light therapy is used in many medical and/or cosmetic areas. The salts according to the invention and the devices containing these salts can therefore be employed for the therapy and/or prophylaxis and/or diagnosis of all diseases and/or in cosmetic applications for which the person skilled in the art considers the use of phototherapy. Besides irradiation, the term phototherapy also includes photodynamic therapy (PDT) and disinfection and sterilisation in general. Phototherapy or light therapy can be used for the treatment of not only humans or animals, but also any other type of living or non-living materials. These include, for example, fungi, bacteria, microbes, viruses, eukaryotes, prokaryotes, foods, drinks, water and drinking water.

The term phototherapy also includes any type of combination of light therapy and other types of therapy, such as, for example, treatment with active compounds. Many light therapies have the aim of irradiating or treating exterior parts of an object, such as the skin of humans and animals, wounds, mucous membranes, the eye, hair, nails, the nail bed, gums and the tongue. However, the treatment or irradiation according to the invention can also be carried out inside an object in order, for example, to treat internal organs (heart, lung, etc.) or blood vessels or the breast.

The therapeutic and/or cosmetic areas of application according to the invention are preferably selected from the group of skin diseases and skin-associated diseases or changes or conditions, such as, for example, psoriasis, skin ageing, skin wrinkling, skin rejuvenation, enlarged skin pores, cellulite, oily/greasy skin, folliculitis, actinic keratosis, precancerous actinic keratosis, skin lesions, sun-damaged and sun-stressed skin, crows' feet, skin ulcers, acne, acne rosacea, scars caused by acne, acne bacteria, photomodulation of greasy/oily sebaceous glands and their surrounding tissue, jaundice, jaundice of the newborn, vitiligo, skin cancer, skin tumours, Crigler-Najjar, dermatitis, atopic dermatitis, diabetic skin ulcers and desensitisation of the skin.

Particular preference is given for the purposes of the invention to the treatment and/or prophylaxis of psoriasis, acne, cellulite, skin wrinkling, skin ageing, icterus and vitiligo.

Further areas of application according to the invention for the compositions and/or devices containing the compositions according to the invention are selected from the group of inflammatory diseases, rheumatoid arthritis, pain therapy, treatment of wounds, neurological diseases and conditions, oedema, Paget's disease, primary and metastasising tumours, connective-tissue diseases or changes, changes in the collagen, fibroblasts and cell level originating from fibroblasts in tissues of mammals, irradiation of the retina, neovascular and hypertrophic diseases, allergic reactions, irradiation of the respiratory tract, sweating, ocular neovascular diseases, viral infections, particularly infections caused by herpes simplex or HPV (human papillomaviruses) for the treatment of warts and genital warts.

Particular preference is given for the purposes of the invention to the treatment and/or prophylaxis of rheumatoid arthritis, viral infections and pain.

Further areas of application according to the invention for the salts and/or devices containing the salts according to the invention are selected from winter depression, sleeping sickness, irradiation for improving the mood, the reduction in pain particularly muscular pain caused by, for example, tension or joint pain, elimination of joint stiffness and the whitening of the teeth (bleaching).

Further areas of application according to the invention for the salts and/or devices containing the salts according to the invention are selected from the group of disinfections. The salts according to the invention and/or the devices according to the invention can be used for the treatment of any type of objects (non-living materials) or subjects (living materials such as, for example, humans and animals) for the purposes of disinfection, sterilisation or preservation. This includes, for example, the disinfection of wounds, the reduction in bacteria, the disinfection of surgical instruments or other articles, the disinfection or preservation of foods, of liquids, in particular water, drinking water and other drinks, the disinfection of mucous membranes and gums and teeth. Disinfection here is taken to mean the reduction in the living microbiological causative agents of undesired effects, such as bacteria and germs.

For the purposes of the above-mentioned phototherapy, devices containing the salts according to the invention preferably emit light having a wavelength between 250 and 1250 nm, particularly preferably between 300 and 1000 nm and especially preferably between 400 and 850 nm.

In a particularly preferred embodiment of the present invention, the salts according to the invention are employed in an organic light-emitting diode (OLED) or an organic light-emitting electrochemical cell (OLEC) for the purposes of phototherapy. Both the OLED and the OLEC can have a planar or fibre-like structure having any desired cross section (for example round, oval, polygonal, square) with a single- or multilayered structure. These OLECs and/or OLEDs can be installed in other devices which comprise further mechanical, adhesive and/or electronic elements (for example battery and/or control unit for adjustment of the irradiation times, intensities and wavelengths). These devices containing the OLECs and/or OLEDs according to the invention are preferably selected from the group comprising plasters, pads, tapes, bandages, cuffs, blankets, caps, sleeping bags, textiles and stents.

The use of the said devices for the said therapeutic and/or cosmetic purpose is particularly advantageous compared with the prior art, since homogeneous irradiation of lower irradiation intensity is possible at virtually any site and at any time of day with the aid of the devices according to the invention using the OLEDs and/or OLECs. The irradiation can be carried out as an inpatient, as an outpatient and/or by the patient themselves, i.e. without initiation by medical or cosmetic specialists. Thus, for example, plasters can be worn under clothing, so that irradiation is also possible during working hours, in leisure time or during sleep. Complex inpatient/outpatient treatments can in many cases be avoided or their frequency reduced. The devices according to the invention may be intended for reuse or be disposable articles, which can be disposed of after use once, twice or three times.

Further advantages over the prior art are, for example, lower evolution of heat and emotional aspects. Thus, newborn being treated owing to jaundice typically have to be irradiated blindfolded in an incubator without physical contact with the parents, which represents an emotional stress situation for parents and newborn. With the aid of a blanket according to the invention comprising the OLEDs and/or OLECs according to the invention, the emotional stress can be reduced significantly. In addition, better temperature control of the child is possible due to reduced heat production of the devices according to the invention compared with conventional irradiation equipment.

It should be pointed out that variations of the embodiments described in the present invention fall within the scope of this invention. Each feature disclosed in the present invention can, unless explicitly excluded, be replaced by alternative features which serve the same, an equivalent or a similar purpose. Thus, each feature disclosed in the present invention should, unless stated otherwise, be regarded as an example of a generic series or as an equivalent or similar feature.

All features of the present invention can be combined with one another in any way, unless certain features and/or steps are mutually exclusive. This applies, in particular, to preferred features of the present invention. Equally, features of non-essential combinations can be used separately (and not in combination).

It should furthermore be pointed out that many of the features, and in particular those of the preferred embodiments of the present invention, should be regarded as inventive themselves and not merely as part of the embodiments of the present invention. Independent protection may be granted for these features in addition or as an alternative to each invention claimed at present.

The teaching regarding technical action disclosed with the present invention can be abstracted and combined with other examples.

The invention is explained in greater detail by the following examples without wishing it to be restricted thereby.

### SYNTHESIS AND WORKING EXAMPLES

The following materials are used in this application.

V1 is a red emitter (DOCI), purchased from Lambda Physik, Germany.

V2 is a blue triplet emitter, synthesised in accordance with WO 2010/089393;

V3 is a green triplet emitter, synthesised in accordance with WO 2007/006380.

V4 and V5 are blue-fluorescent emitters, product name C-2284 and 1-6076 from Invitrogen Corporation.

V6 is a green-fluorescent emitter, product name F-1907 from Invitrogen Corporation.

V7 is an infrared absorber, synthesised in accordance with WO 2010/095676 A1.

Z-907 is a dye from Sigma-Aldrich, with product No. 703168.

M1 is a matrix compound for fluorescent emitters, synthesised in accordance with WO 2007/065678.

M2 is a soluble matrix compound for phosphorescent emitters, synthesised in accordance with WO 2005/003253.

E1-E4 are the compounds according to the invention.

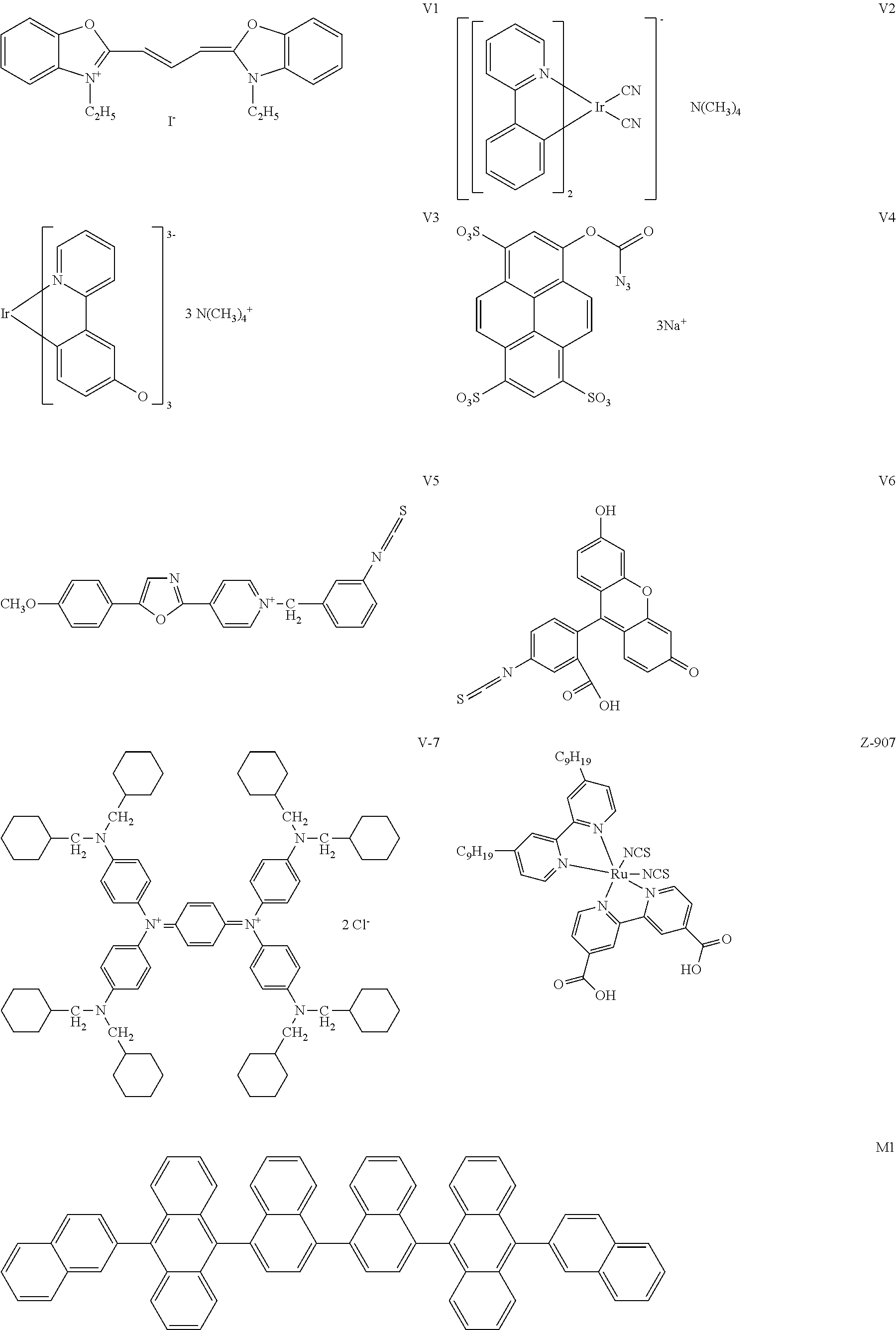

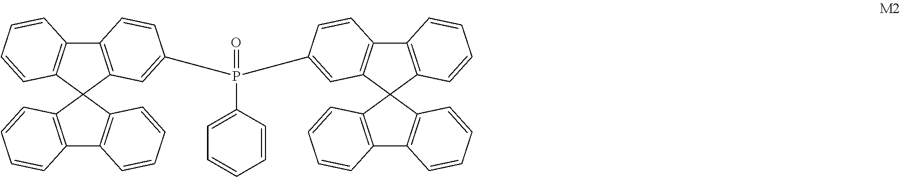 [SMILES: O=P(c1ccccc1)(c1ccc2c(c1)C1(c3ccccc3-c3ccccc31)c1ccccc1-2)c1ccc2c(c1)C1(c3ccccc3-c3ccccc31)c1ccccc1-2]

Structures of the compounds according to the invention:

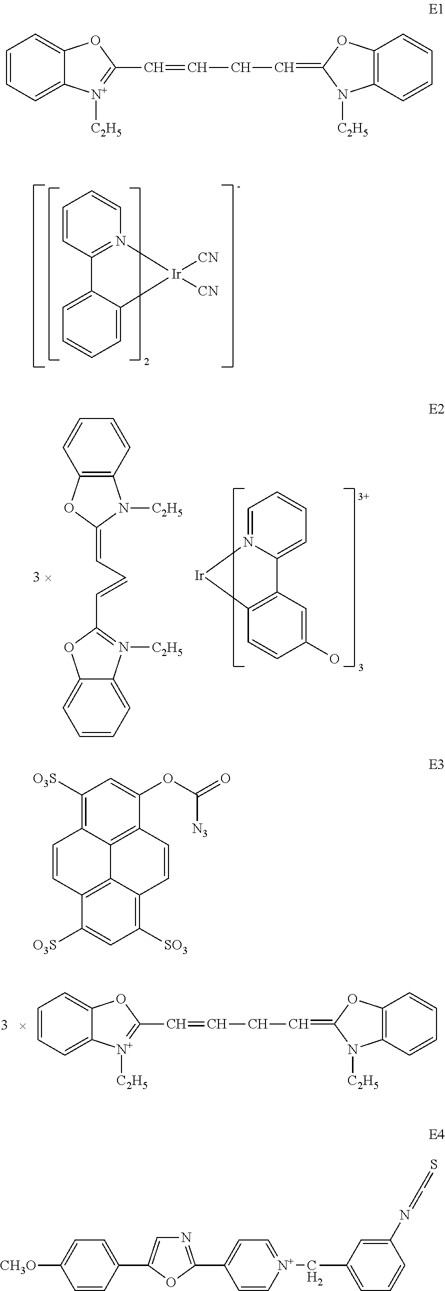

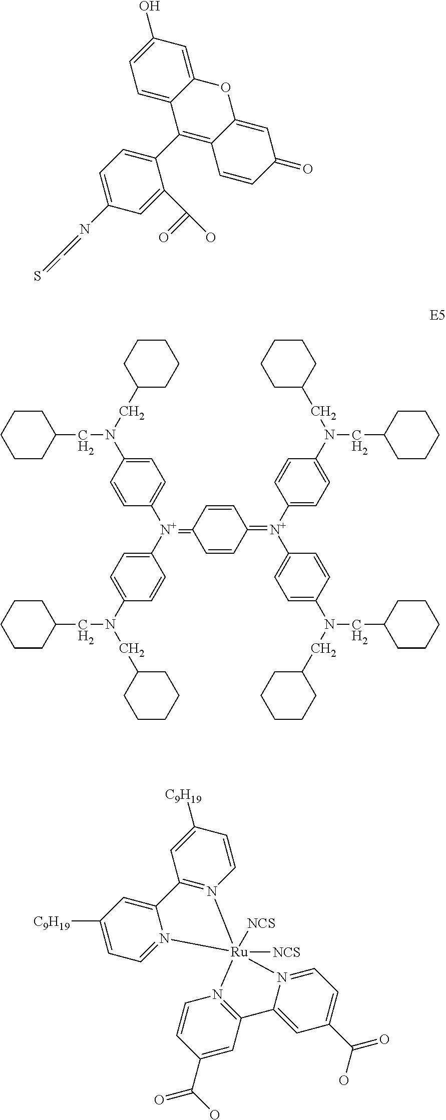

### Example 1

### Preparation of salts E1, E2, E3, and E4

A solution of 4.46 g (10 mmol) of 3-ethyl-2-[3-(3-ethyl-2-benzoxazolinylidene)propenyl]benzoxazolium iodide [905-96-4] (compound 2) in 100 ml of acetonitrile is added dropwise with vigorous stirring to a solution of 7.98 g (10 mmol) of tetra-n-butylammonium bis(cyano-κC)bis[2-(2-pyridinyl-κN)phenyl-κC]iridate(III) [577751-00-9] (compound 1) in 100 ml of acetonitrile, and the mixture is then stirred at room temperature for 30 min. The reaction mixture is subsequently concentrated in vacuo to a volume of about 50 ml, and 300 ml of a water/methanol mixture (1:2, vv) are then added dropwise. The mixture is stirred at room temperature for a further 1 h, the precipitated solid is filtered off with suction, and the latter is recrystallised three times from methanol. Yield: 5.10 g (5.8 mmol), 58%. Purity app. 99.5% (¹H-NMR).

The following compounds can be prepared analogously:

| Ex. | Comp. 1 | Comp. 2 | Product | Yield |
|---|---|---|---|---|
| E2 | 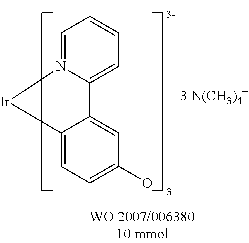 | 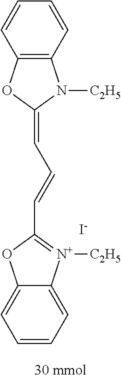 [SMILES: CCN1/C(=C\C=C\c2oc3ccccc3[n+]2CC)Oc2ccccc21 ; [I-]] | 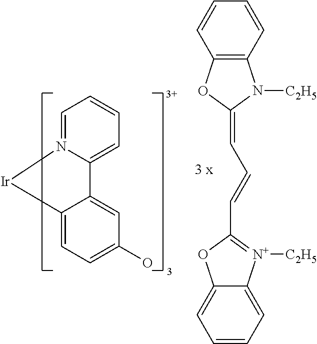 | 61% |
| E3 | 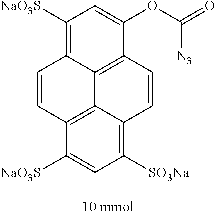 | 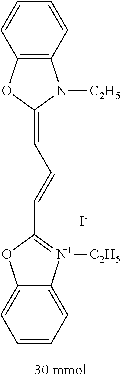 [SMILES: CCN1/C(=C\C=C\c2oc3ccccc3[n+]2CC)Oc2ccccc21 ; [I-]] | 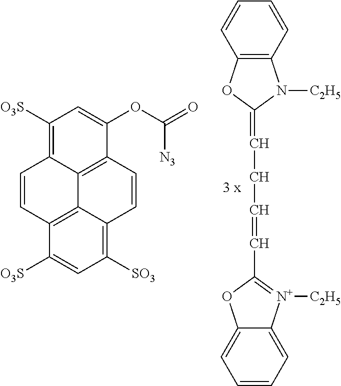 | 45% |
| E4 | 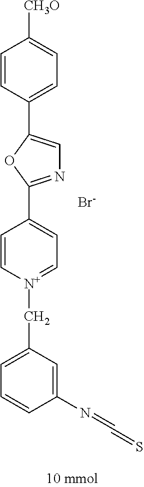 | 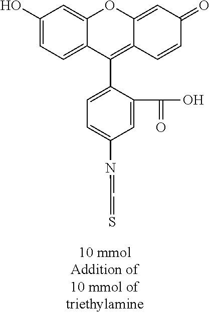 [SMILES: O=C(O)c1cc(N=CC=S)ccc1-c1c2ccc(=O)cc-2oc2cc(O)ccc12] | 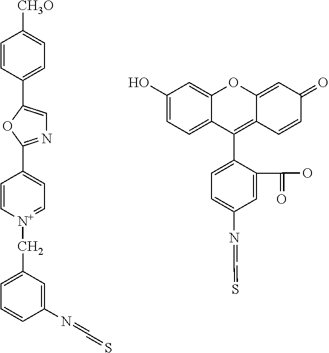 | 39% |
| E5 | V7 | Z-907 | E5 | 28% |
| | 10 mmol | 10 mmol | | |

### Example 2

### Production and Characterisation of Organic Electroluminescent Devices Containing Compounds E1-E4 According to the Invention

The production of an organic light-emitting diode from solution has already been described many times in the literature (for example in WO 2004/037887 A2). In order to explain the present invention by way of example, triplet OLEDs having various combinations of E1-E4 in the matrix is produced by spin coating.

A typical OLED device has the structure:

ITO/HIL/interlayer/EML/cathode, where HIL is also referred to as buffer layer.

To this end, use is made of substrates from Technoprint (soda-lime glass) to which the ITO structure (indium tin oxide, a transparent, conductive anode) is applied.

The substrates are cleaned with DI water and a detergent (Deconex 15 PF) in a clean room and then activated by UV/ozone plasma treatment. An 80 nm layer of PEDOT (PEDOT is a polythiophene derivative (Baytron P VAI 4083sp.) from H. C. Starck, Goslar, which is supplied as an aqueous dispersion) is then applied as buffer layer by spin coating, likewise in the clean room. The spin rate required depends on the degree of dilution and the specific spin-coater geometry (typical for 80 nm: 4500 rpm). In order to remove residual water from the layer, the substrates are dried by heating on a hotplate at 180° C. for 10 minutes. Firstly 20 nm of an interlayer (typically a hole-dominated polymer, here HIL-012 from Merck KGaA) and then 80 nm of the emitting layers (EML for emissive layer) are then applied from solutions (concentration 20 g/l in chlorobenzene, the compositions for the various EMLs, and the concentrations thereof are listed in Table 1) under an inert-gas atmosphere (nitrogen or argon). All EML layers are dried by heating at 60° C. for at least 30 minutes. The Ba/Al cathode is then applied by vapour deposition (high-purity metals from Aldrich, particularly barium 99.99% (Order No. 474711); vapour-deposition units from Lesker or others, typical vacuum level 5×10⁻⁶ mbar). In order to protect, in particular, the cathode against air and atmospheric moisture, the device is finally encapsulated and then characterised.

TABLE 1
The EML compositions in various OLEDs

| | EML composition | | Concentration |
|---|---|---|---|
| Device | [wt %] | Solvent | [g/l] |
| OLED1 | M2: 5% V1 | Chlorobenzene | 20 |
| OLED2 | M2: 5% V2 | Chlorobenzene | 20 |
| OLED3 | M2: 5% V3 | Chlorobenzene | 20 |
| OLED4 | M2: 5% E1 | Chlorobenzene | 20 |
| OLED5 | M2: 5% E2 | Chlorobenzene | 20 |
| OLED6 | M1: 5% V1 | Chlorobenzene | 20 |
| OLED7 | M1: 5% V5 | Chlorobenzene | 20 |
| OLED8 | M1: 5% V6 | Chlorobenzene | 20 |
| OLED9 | M1: 5% E3 | Chlorobenzene | 20 |
| OLED10 | M1: 5% E4 | Chlorobenzene | 20 |

To this end, the devices are clamped into holders manufactured specifically for the substrate size and provided with spring contacts. A photodiode with eye response filter can be attached directly to the measurement holder in order to exclude influences by extraneous light.

The voltages are typically increased from 0 to max. 20 V in 0.2 V steps and reduced again. For each measurement point, the current through the device and the photocurrent obtained is measured by the photodiode. In this way, the IVL data of the test devices are obtained. Important parameters are the maximum efficiency measured (“eff.” in cd/A) and the voltage U₁₀₀ required for 100 cd/m².

In order, in addition, to know the colour and the precise electroluminescence spectrum of the test devices, the voltage required for 100 cd/m² is applied again after the first measurement, and the photodiode is replaced by a spectrum measurement head. This is connected to a spectrometer (Ocean Optics) by an optical fibre. The colour coordinates (CIE: Commission International de I'éclairage, standard observer from 1931) can be derived from the measured spectrum.

The results obtained on use of emitters E1 to E4 in OLEDs are summarised in Table 2.

TABLE 2

| | Uon | U(100) | CIE @ | EQE @ |
|---|---|---|---|---|
| Device | [V] | [V] | 100 cd/m² | max. eff. |
| OLED1 | 4.6 | 6.1 | 0.66/0.36 | 0.5% |
| OLED2 | 4.3 | 5.9 | 0.19/0.31 | 0.9% |
| OLED3 | 3.3 | 4.9 | 0.33/0.63 | 1.1% |
| OLED4 | 4.0 | 5.7 | 0.66/0.36 | 1.9% |
| OLED5 | 3.5 | 5.0 | 0.66/0.35 | 2.2% |
| OLED6 | 3.1 | 4.0 | 0.65/0.36 | 1.9% |
| OLED7 | 3.9 | 4.7 | 0.18/0.33 | 1.3% |
| OLED8 | 3.5 | 4.9 | 0.34/0.65 | 2.2% |
| OLED9 | 2.9 | 3.9 | 0.66/0.35 | 2.9% |
| OLED10 | 3.0 | 4.6 | 0.33/0.66 | 3.8% |

As can be seen from the results, OLED4 and OLED5 represent a significant improvement over OLED1-OLED3 with respect to the efficiency and operating voltage. OLED1-OLED3 are OLEDs comprising individual emitters V1-V3. OLED4 and OLED5 are OLEDs comprising a salt according to the invention, where two emitters have been doped into the matrix so closely that energy transfer from E1 to E2 can take place very efficiently. The same also applies to OLED9 and OLED10 compared with OLED6-OLED8.

Furthermore, the table also reveals that OLED9 & OLED10, in which both emitters are not metal complex emitters, give a better EQE and a better operating voltage compared with OLED5 & OLED6, in which the salts consist of a metal complex and a fluorescent emitter.

Based on the present technical teaching according to the invention, further optimisations can be achieved by means of various possibilities without being inventive. Thus, a further optimisation can be achieved, for example, through the use of another matrix or mixed matrices in the same or another concentration.

### Example 3

### Absorption Spectra of V7, Z-907 and E5

The absorption spectra of V7, Z-907 and E5 are measured in ethanol solution with a concentration of about 0.3 nM. Compared with Z-907, E5 exhibits a significant improvement in the near infrared region (650 to 1000 nm).

1-22. (canceled)

23. A salt in which both at least one cation and also at least one anion is an emitter compound or a dye compound, where one emitter compound is a fluorescent emitter compound.

24. The salt according to claim 23, in which all emitter compounds are non-metal compounds.

25. The salt according to claim 23, in which all emitter compounds are fluorescent emitter compounds.

26. The salt according to claim 23, in which either the cation or the anion is a metal-complex dye compound, and the respective other is a fluorescent emitter compound.

27. The salt according to claim 23, which is in the form [E₁]^{+m}[E₂]^{-m}, where [E₁]^{+m} is an emitter compound having the charge +m and [E₂]^{-m} is an emitter compound having the charge −m, where m is equal to 1 or 2.

28. The salt according to claim 27, in which [E₁]^{+m} is a phosphorescent emitter compound T^{+m} and [E₂]^{-m} is a fluorescent emitter compound S-m.

29. The salt according to claim 28, in which the phosphorescent emitter compound is a metal-ligand coordination compound.

30. The salt according to claim 27, in which [E₁]^{+m} and [E₂]^{-m} are fluorescent emitter compounds S₁^{+m} and S₂^{-m}.

31. The salt according to claim 27, in which m is equal to 1.

32. The salt according to claim 23, which is in the form [E₃]^{k}[E₁]^{p}[E₂]^{k}, where [E₁]^{p} is an emitter compound having the charge p and [E₂]^{k} and [E₃]^{k} are emitter compounds having the charge k, where and p+2k=0, and k equals to ±1 or ±2.

33. The salt according to claim 32, in which [E₂]^{k} and/or [E₃]^{k} is (are) (a) fluorescent emitter compound.

34. The salt according to claim 32, in which k is equal to 1.

35. The salt according to claim 23, in which the emission band of an emitter compound is in a wavelength range which overlaps with the wavelength range of the absorption band of another emitter compound.

36. The salt according to claim 32, in which the maximum of the emission band of [E₁]^{p} is in the wavelength region of blue light, and the maxima of the emission bands of [E₂]^{k} and [E₃]^{k} are in the wavelength region of green or red light.

37. A process for the preparation of the salt according to claim 23 comprising the following steps:
(a) proving a multiplicity of salts, where each salt contains only one emitter compound as cation or anion and where at least one emitter compound has a positive charge and at least one emitter compound has a negative charge;
(b) respective dissolution of the salts provided in a polar solvent, giving a multiplicity of solutions corresponding to the number of salts;
(c) mixing of the solutions; and
(d) crystallizing a salt which contains the at least one emitter compound having the positive charge as a cation and the at least one emitter compound having the negative charge as an anion.

38. The process according to claim 37, in which two salts [E₁]⁺Y⁻ and [E₂]⁻X⁺ are provided in step (a), so that a salt [E₁]⁺[E₂]⁻ is obtained in step (d), where Y⁻ and X⁺ are corresponding counterions which together form a salt X⁺Y⁻ which is more readily soluble than [E₁]⁺[E₂]⁻.

39. The process according to claim 38, in which three salts [E₁]²⁺[2Y]⁻, [E₂]⁻[X]⁺ and [E₃]⁻[X]⁺ are provided in step (a), so that a salt [E₃]⁻[E₁]²⁺[E₂]⁻ is obtained in step (d), where Y⁻ and X⁺ are corresponding counterions which together form a salt X⁺Y⁻ which is more readily soluble than [E₃]⁻[E₁]²⁺[E₂]⁻.

40. An electronic device comprising the salt according to claim 23.

41. An organic electroluminescent device comprising the salt according to claim 23.

42. The electronic device according to claim 40, wherein the device is an organic integrated circuit, an organic field-effect transistor, an organic thin-film transistor, an organic solar cell, a dye-sensitized organic solar cell, an organic optical detector, an organic photoreceptor, an organic field-quench device, an organic laser diode or an organic plasmon emitting device.

43. A formulation comprising the salt according to claim 23 dissolved, dispersed or emulsified in at least one solvent.

44. A process for the therapy, prophylaxis and/or diagnosis of diseases and/or cosmetic conditions which comprises utilizing the salt according to claim 23.
